(19) United States
(12) Patent Application Publication
(10) Pub. No.: US 2026/0347336 A1
(43) Pub. Date: Nov. 5, 2026

(54) PRODRUGS, FORMULATIONS AND METHODS THEREOF

(71) Applicant: OCULAR THERAPEUTIX, INC., Bedford, MA (US)

(72) Inventors: Peter JARRETT, Bedford, MA (US); Rami EL-HAYEK, Bedford, MA (US)

(21) Appl. No.: 19/117,581

(22) PCT Filed: Oct. 13, 2023
(86) PCT No.: PCT/US2023/035121

§ 371 Date: Apr. 1, 2025

Related U.S. Application Data
(60) Provisional application No. 63/416,292, filed on Oct. 14, 2022.

(51) Int. Cl. C07D 401/06; A61K 31/4439

(52) U.S. Cl. CPC C07D 401/06; A61K 31/4439

(57) ABSTRACT

In certain embodiments, the invention is directed to a compound of formula (I): wherein; X¹ is selected from N or N⁺Y¹; X² is selected from NH or NY²; X³ is selected from NH or NY³; Y1 is selected from —CH₂OCO(OCH₂CH₂)n¹OM¹; or —CH₂OCO(CH₂CH₃O)n^{1a}Z¹; or —CH₂OCO(CH₂)n^{1b}COOH; Y² is selected from —CH₂OCO(OCH₂CH₂)n²OM²; or —CH₂OCO(CH₂CH₂O)n^{2a}Z²; or —CH₂OCO(CH₂)n^{2b}COOH; Y³ is selected from —CH₂OCO(OCH₂CH₂)n³OM³; or —CH₂O—CO(CH₂CH₂O)n^{3a}Z³; or —CH₂OCO(CH₂)n^{3b}COOH; n¹, n^{1a}, n^{1b}, n², n^{2a}, n^{2b}, n³, n^{3a}, and n^{3b} are independently 0 or an integer from 1 to 8; M¹, M², M³, Z¹, Z² and Z³ are independently selected from H, optionally substituted C₁₋₆alkyl and optionally substituted aryl; wherein at least one of X¹, X² and X³ is not N or NH; wherein at least one of Y¹, Y² or Y³ are the respective ·—CH₂OCO(CH₂CH₂O)nZ; and pharmaceutically acceptable salts thereof.

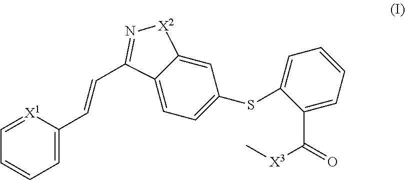

### FIELD OF THE INVENTION

The present application is directed to prodrugs, pharmaceutical compositions comprising the prodrugs and corresponding methods of treatment.

### BACKGROUND

Tyrosine kinase inhibitors were developed as chemotherapeutics that inhibit signaling of receptor tyrosine kinases (RTKs), which are a family of tyrosine protein kinases. RTKs span the cell membrane with an intracellular (internal) and extracellular (external) portion. Upon ligand binding to the extracellular portion, receptor tyrosine kinases dimerize and initiate an intracellular signaling cascade driven by autophosphorylation using the coenzyme messenger adenosine triphosphate (ATP). Many of the RTK ligands are growth factors such as VEGF. VEGF relates to a family of proteins binding to VEGF-receptor (VEGFR) types, i.e. VEGFR1-3 (all RTKs), thereby inducing angiogenesis. VEGF-A, which binds to VEGFR2, is the target of the anti-VEGF drugs described above. Besides VEGFR1-3 several other RTKs are known to induce angiogenesis such as platelet-derived growth factor receptor (PDGFR) activated by PDGF or stem cell growth factor receptor/type III receptor tyrosine kinase (c-Kit) activated by stem cell factor.

The TKI axitinib is used alone to treat advanced renal cell carcinoma (RCC, a type of cancer that begins in the cells of the kidneys) in people who have not been treated successfully with another medication. Axitinib is used in combination with avelumab or pembrolizumab to treat advanced renal cell carcinoma.

Some TKIs have been evaluated for the treatment of age-related macular degeneration (AMD) via different administration routes, including pazopanib (GlaxoSmithKline: NCT00463320), regorafenib (Bayer: NCT02348359), and PAN90806 (PanOptica: NCT02022540) all administered as eye drops, as well as X-82, an oral TKI (Tyrogenex; NCT01674569, NCT02348359). However, topically applied eye drops result in poor penetration into the vitreous and limited distribution to the retina due to low solution concentration of TKIs, which tend to have low water solubility, and short residence time of the TKIs on the ocular surface. Moreover, drug concentration upon topical administration is difficult to control due to wash out or user error. Furthermore, systemic administration of TKIs is not practicable, as high doses are required to achieve effective concentrations of the drug in the eye and particularly at the desired tissue. This leads to unacceptable side effects due to high systemic exposure. In addition, drug concentrations are difficult to control. Alternatively, intravitreal injections of TKI suspensions have been performed. However, this way of administration results in rapid clearance of the drug and therefore injections have to be repeated frequently, such as on a daily or at least a monthly basis. In addition, several TKIs are poorly soluble which leads to the formation of aggregates upon intravitreal injection, which can migrate or settle onto the retina and lead to local contact toxicity and holes, such as macular or retinal holes.

Thus, there is a need in the art for new compounds, pharmaceutical compositions and methods of treatment such as to treat disease states with TKI therapy such as renal cell carcinoma and ocular diseases such as AMD, diabetic macular edema (DME) and retinal vein occlusion (RVO).

All references cited herein are incorporated by reference in their entireties for all purposes.

### OBJECTS AND SUMMARY

It is an object of certain embodiments of the invention to provide to compounds for the treatment of diseases or conditions.

It is an object of certain embodiments of the invention to provide pharmaceutical compositions comprising the compounds disclosed herein.

It is an object of certain embodiments of the invention to provide methods of treatment with the compounds and pharmaceutical compositions disclosed herein.

It is an object of certain embodiments of the invention to provide methods of preparing the compounds and pharmaceutical compositions disclosed herein.

It is an object of certain embodiments of the invention to provide axitinib prodrugs that are more soluble than axitinib, e.g., being at least 2 times, 10 times, 25 times, 50 times, 75 times, 100 times, 150 times, 200 times, 250 times, 500 times or 1000 times more soluble or a range of any of these values, e.g., from 2 to 200 times more soluble, from 10 to 100 more times soluble or from 50 to about 150 times more soluble.

It is an object of certain embodiments of the invention to provide methods of adjusting the release of an active agent from a hydrogel or organogel comprising including a prodrug in a hydrogel or organogel dosage form.

It is an object of certain embodiments of the invention to provide prodrugs of axitinib to treat diseases or conditions with axitinib therapy.

It is an object of certain embodiments of the invention to provide prodrugs of axitinib in a hydrogel or xerogel (which converts to a hydrogel in vivo) matrix to form an implant for sustained delivery of the prodrug to the local tissue, where it is then converted to axitinib enzymatically. The increased solubility of the prodrug functions to speed the release of the drug from the hydrogel implant compared to the more hydrophobic active drug form, axitinib.

In certain embodiments, the present invention is directed to a compound of formula. I:

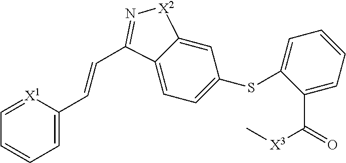

wherein:
X¹ is selected from N or N⁺Y¹;
X² is selected from NH or NY²;
X³ is selected from NH or NY³;
Y¹ is selected from —CH₂OCO(OCH₂CH₂)n¹OM¹; or —CH₂OCO(CH₂CH₂O)n^{1a}Z¹; or —CH₂OCO(CH)n^{1b}COOH;
Y² is selected from —CH₂OCO(OCH₂CH₂)n²OM²; or —CH₂OCO(CH₂CH₂O)n^{2a}Z²; or —CH₂OCO(CH)n^{2b}COOH;
Y³ is selected from CH₂OCO(OCH₂CHO)n³OM³; or —CH₂OCO(CH₂CH₂O)n^{3a}Z³; or —CH₂OCO(CH₂)n^{3b}COOH;
n¹, n^{1a}, n^{1b}, n², n^{2a}, n^{2b}, n³, n^{3a} and n^{3b} are independently 0 or an integer from 1 to 8;
M¹, M², M³, Z¹, Z² and Z³ are independently selected from H, optionally substituted C₁₋₆alkyl and optionally substituted aryl;
wherein at least one of X¹, X² and X³ is not N or NH;
wherein at least one of Y¹, Y² or Y³ are the respective —CH₂OCO(CH₂CH₂O)nZ; and pharmaceutically acceptable salts thereof.

In other embodiments Y¹, Y² and Y³ are independently selected from the respective —(CH₂)p¹OCO(O(CH₃)p²)n¹OM, or —(CH₂)p^{1a}OCO((CH₂)p²O)n¹(CH2)Z; or —(CH₂)p¹OCO(CH₂)q¹COOH; wherein p¹, p^{1a} and p² are independently selected from an integer from 1 to 4 and q¹ is independently selected from an integer from 0 to 4.

The term “sustained release, biodegradable drug-delivery system” as used herein refers to an object that contains an active agent and that is administered, e.g., as an implant, to a patient's body where it remains for a certain period of time while it releases the active agent into the surrounding environment. A drug-delivery system can be of any predetermined shape (e.g., rod, spherical, oblate, ellipsoidal, disc, tube, hemispherical, or irregularly shaped) before being inserted or administered, which shape may be maintained to a certain degree upon placing the system into the desired location, although dimensions of the system (e.g., length and/or diameter) may change after administration due to hydration and/biodegradation as further disclosed herein. The drug-delivery system can be designed to be biodegradable over the course of time (as disclosed below), and thus may thereby soften, change its shape and/or decrease in size, and in the end might be eliminated either by dissolution or disintegration.

The term “biodegradable” refers to a material or object (such as the drug-delivery system according to the present invention) which becomes degraded in vivo, i.e., when placed in the human or animal body or in vitro when immersed in an aqueous solution under physiological conditions such as pH 7.2-7.4 at 37 °C. In the context of the present invention, as disclosed in detail herein below, the drug-delivery system comprising the organogel within which an active agent is contained, slowly biodegrades over time once administered or deposited in the human or animal body. In certain embodiments, biodegradation takes place at least in part via ester hydrolysis in the aqueous environment of the body. Biodegradation may take place by hydrolysis or enzymatic cleavage of the covalent crosslinks and/or within the polymer units. The drug-delivery system slowly softens and disintegrates, resulting in clearance through physiological pathways. In certain embodiments, the organogel of the present invention retains its shape over extended periods of time (e.g., about 1 month, 3 months or 6 months). In certain embodiments, the shape is maintained due to covalent crosslinking of the polymer components forming the organogel, e.g., until the active agent or at least a major amount (e.g., at least 50%, at least 75% or at least 90%) thereof has been released.

An “organogel” in the present invention is a solid or semi-solid system forming a covalently crosslinked three-dimensional network of one or more hydrophilic or hydrophobic natural or synthetic polymers (as disclosed herein) that include a hydrophobic organic liquid as disclosed herein. Thus, in the present invention “organogels” are limited to so-called chemical organogels, wherein the intermolecular interaction between organogelator molecules is a chemical linkage (e.g., covalent bond) that is formed during gelation by chemical reactions inducing crosslinking. The “organogel” as used herein refers to a three-dimensional polymer network of at least two precursors/gelators/precursors that are covalently cross-linked with each other in the presence of a hydrophobic organic liquid and optionally an organic solvent and comprising the hydrophobic organic liquid contained within the covalently crosslinked polymer network.

The term “polymer(ic) network” describes a structure formed of polymer chains (of the same or different molecular structure and of the same or different molecular weight) that are covalently cross-linked with each other. The types of polymers suitable for the purposes of the present invention are disclosed herein below. The term “polymer(ic) network” is used interchangeably with the term “matrix”

For the purpose of the present disclosure, the term “alkyl” as used by itself or as part of another group refers to a straight- or branched-chain aliphatic hydrocarbon containing one to twelve carbon atoms (i.e., C₁₋₁₂ alkyl) or the number of carbon atoms designated (i.e., a C₁ alkyl such as methyl, a C₂ alkyl such as ethyl, a C₃ alkyl such as propyl or isopropyl, etc.). In one embodiment, the alkyl group is chosen from a straight chain C₁₋₁₀ alkyl group. In another embodiment, the alkyl group is chosen from a branched chain C₁₋₁₀ alkyl group. In another embodiment, the alkyl group is chosen from a straight chain C₁₋₆ alkyl group. In another embodiment, the alkyl group is chosen from a branched chain C₁₋₆ alkyl group. In another embodiment, the alkyl group is chosen from a straight chain C₁₋₄ alkyl group. In another embodiment, the alkyl group is chosen from a branched chain C₁₋₄ alkyl group. In another embodiment, the alkyl group is chosen from a straight or branched chain C₂₋₄ alkyl group. Non-limiting exemplary C₁₋₁₀ alkyl groups include methyl, ethyl, propyl, isopropyl, butyl, sec-butyl, tert-butyl, iso-butyl, 3-pentyl, hexyl, heptyl, octyl, nonyl, decyl, and the like. Non-limiting exemplary C₁₋₄ alkyl groups include methyl, ethyl, propyl, isopropyl, butyl, sec-butyl, tert-butyl, and iso-butyl.

For the purpose of the present disclosure, the term “optionally substituted alkyl” as used by itself or as part of another group means that the alkyl as defined above is either unsubstituted or substituted with one, two, or three substituents independently chosen from nitro, haloalkoxy, aryloxy, aralkyloxy, alkylthio, sulfonamido, alkylcarbonyl, arylcarbonyl, alkylsulfonyl, arylsulfonyl, ureido, guanidino, carboxy, carboxyalkyl, cycloalkyl, and the like. In one embodiment, the optionally substituted alkyl is substituted with two substituents. In another embodiment, the optionally substituted alkyl is substituted with one substituent. Non-limiting exemplary optionally substituted alkyl groups include —CH₂CH₂NO₂, —CH₂CH₂CO₂H, —CH₂CH₂SO₂CH₂, —CH₂CH₂COPh, —CH₂C₆H₁₁, and the like.

For the purpose of the present disclosure, the term “aryl” as used by itself or as part of another group refers to a monocyclic or bicyclic aromatic ring system having from six to fourteen carbon atoms (i.e., C₆₋₁₄ aryl). Non-limiting exemplary aryl groups include phenyl (abbreviated as “Ph”), naphthyl, phenanthryl, anthracyl, indenyl, azulenyl, biphenyl, biphenylenyl, and fluorenyl groups. In one embodiment, the aryl group is chosen from phenyl or naphthyl.

For the purpose of the present disclosure, the term “optionally substituted aryl” as used herein by itself or as part of another group means that the aryl as defined above is either unsubstituted or substituted with one to five substituents independently chosen from halo, nitro, cyano, hydroxy, amino, alkylamino, dialkylamino, haloalkyl, hydroxyalkyl, alkoxy, haloalkoxy, aryloxy, aralkyloxy, alkylthio, carboxamido, sulfonamido, alkylcarbonyl, arylcarbonyl, alkylsulfonyl, arylsulfonyl, ureido, guanidino, carboxy, carboxyalkyl, alkyl, cycloalkyl, alkenyl, alkynyl, aryl, heteroaryl, heterocyclo, alkoxyalkyl, (amino)alkyl, hydroxyalkylamino, (alkylamino)alkyl, (dialkylamino)alkyl, (cyano)alkyl, (carboxamido)alkyl, mercaptoalkyl, (heterocyclo)alkyl, or (heteroaryl)alkyl. In one embodiment, the optionally substituted aryl is an optionally substituted phenyl. In one embodiment, the optionally substituted phenyl has four substituents. In another embodiment, the optionally substituted phenyl has three substituents. In another embodiment, the optionally substituted phenyl has two substituents. In another embodiment, the optionally substituted phenyl has one substituent. Non-limiting exemplary substituted aryl groups include 2-methylphenyl, 2-methoxyphenyl, 2-fluorophenyl, 2-chlorophenyl, 2-bromophenyl, 3-methylphenyl, 3-methoxyphenyl, 3-fluorophenyl, 3-chlorophenyl, 4-methylphenyl, 4-ethylphenyl, 4-methoxyphenyl, 4-fluorophenyl, 4-chlorophenyl, 2,6-di-fluorophenyl, 2,6-di-chlorophenyl, 2-methyl, 3-methoxyphenyl, 2-ethyl, 3-methoxyphenyl, 3,4-di-methoxyphenyl, 3,5-di-fluorophenyl 3,5-di-methylphenyl, 3,5-dimethoxy, 4-methylphenyl, 2-fluoro-3-chlorophenyl, and 3-chloro-4-fluorophenyl. The term optionally substituted aryl is meant to include groups having fused optionally substituted cycloalkyl and fused optionally substituted heterocyclo rings. Examples include:

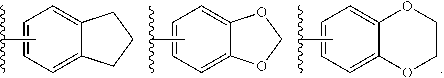

The term “pharmaceutically acceptable salt,” as used herein, can include, but are not limited to, include, inorganic acid salts such as hydrochloride, hydrobromide, sulfate, phosphate and the like, organic acid salts such as formate, acetate, trifluoroacetate, maleate, tartrate and the like; sulfonates such as methanesulfonate, benzenesulfonate, p-toluenesulfonate, and the like; and metal salts such as sodium salt, potassium salt, cesium salt and the like; alkaline earth metals such as calcium salt, magnesium salt and the like, organic amine salts such as triethylamine salt, pyridine salt, picoline salt, ethanolamine salt, triethanolamine salt, dicyclohexylamine salt, N,N′-dibenzylethylenediamine salt and the like. In certain embodiments, the therapeutically effective agent is free base

### BRIEF DESCRIPTION OF THE DRAWINGS

FIG. 1 depicts synthetic schemes of prodrugs of Example 1 and Example 2.

FIG. 2 depicts the 1H-NMR of compound 6 (Int-1) of Example 1

FIG. 3 depicts the 1H-NMR of Example 1.

FIGS. 4 and 5 depict the LCMS of Example 1.

FIG. 6 depicts the HPLC of Example 1.

FIG. 7 depicts stability data of Example 8.

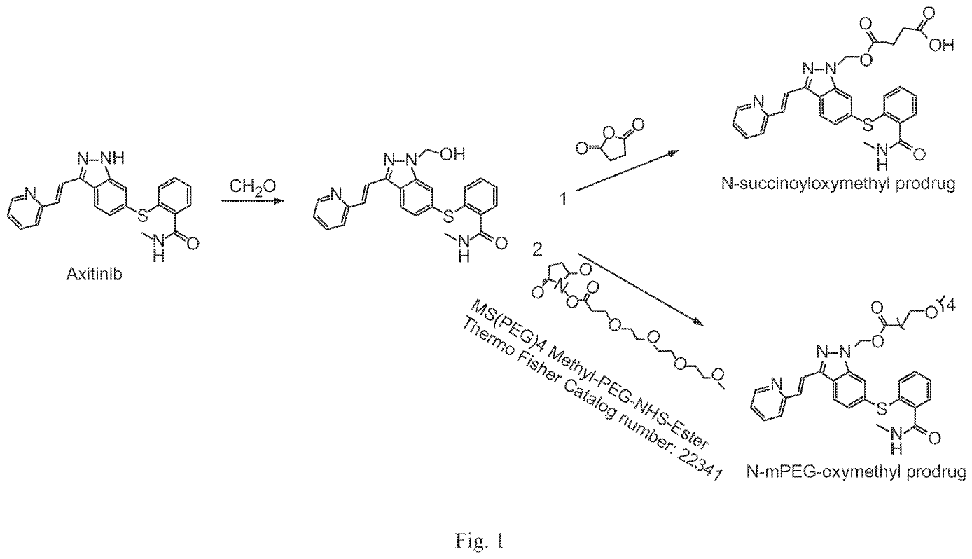

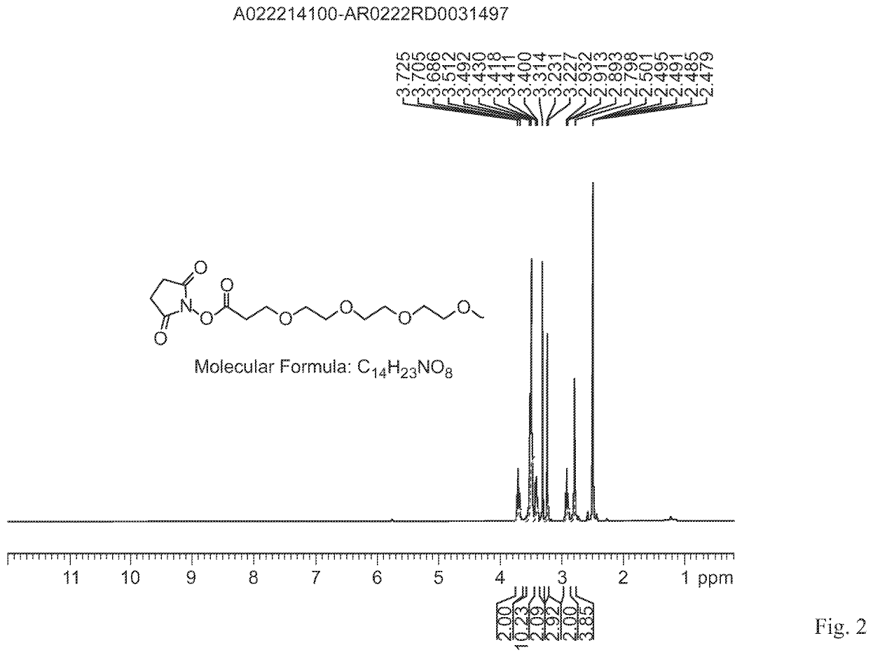

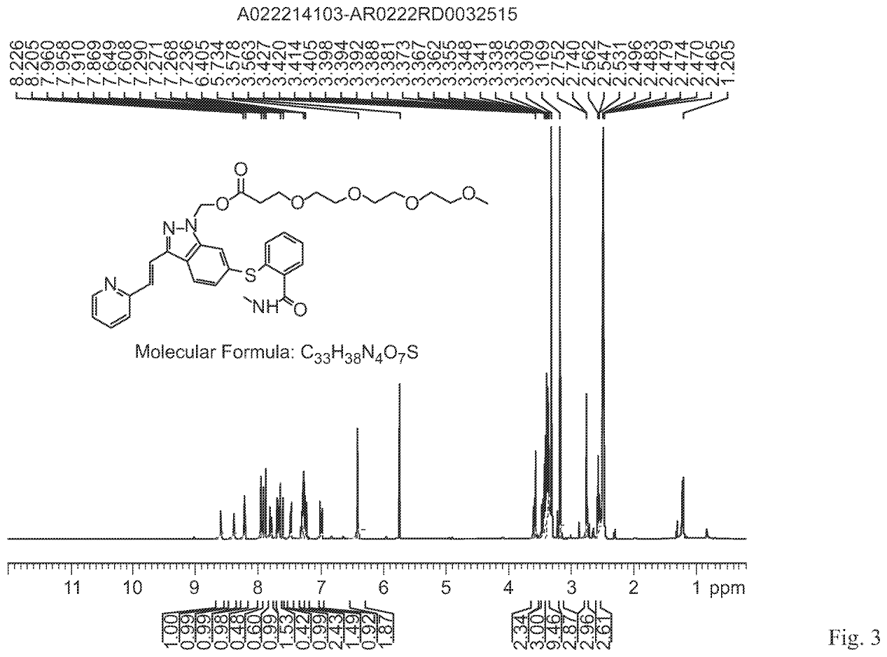

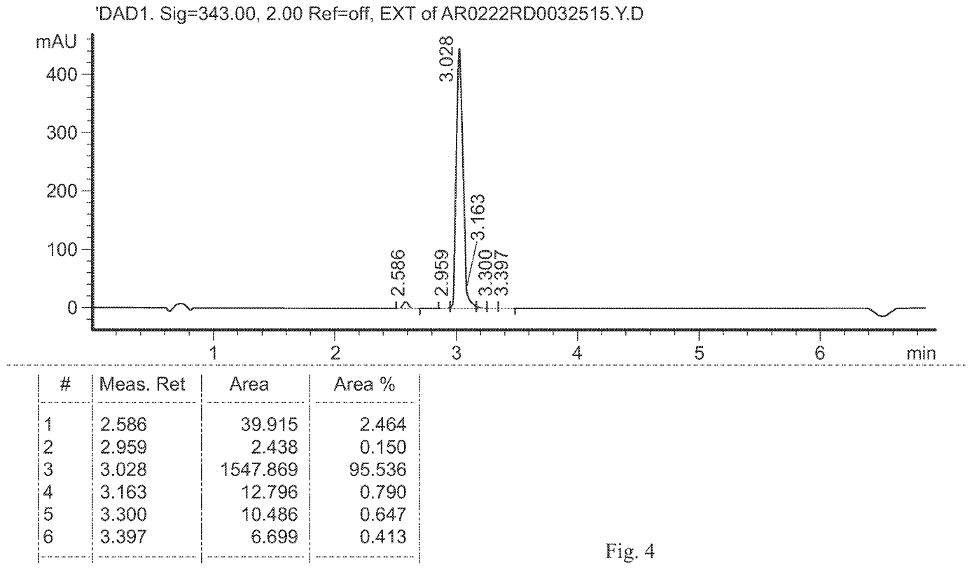

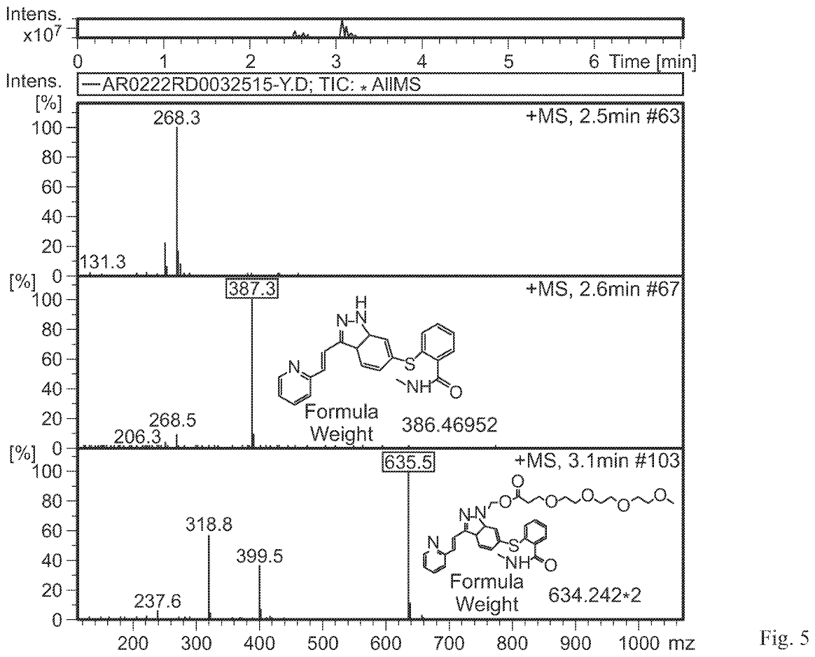

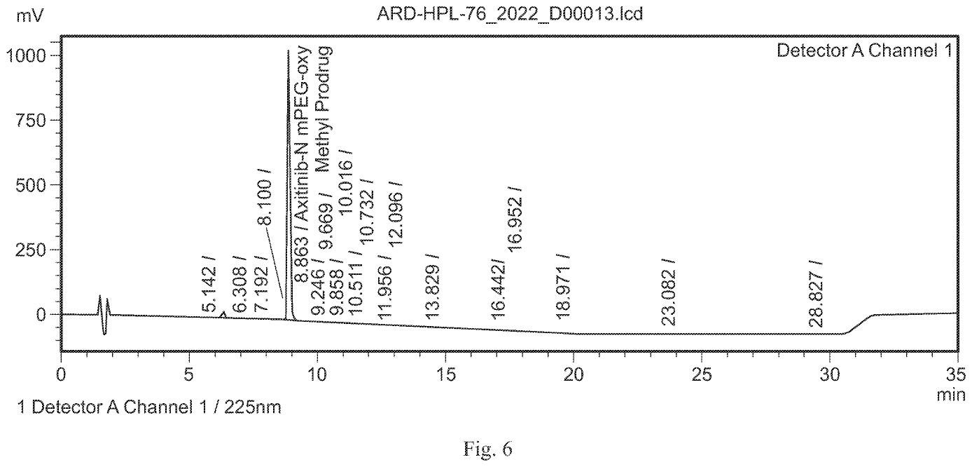

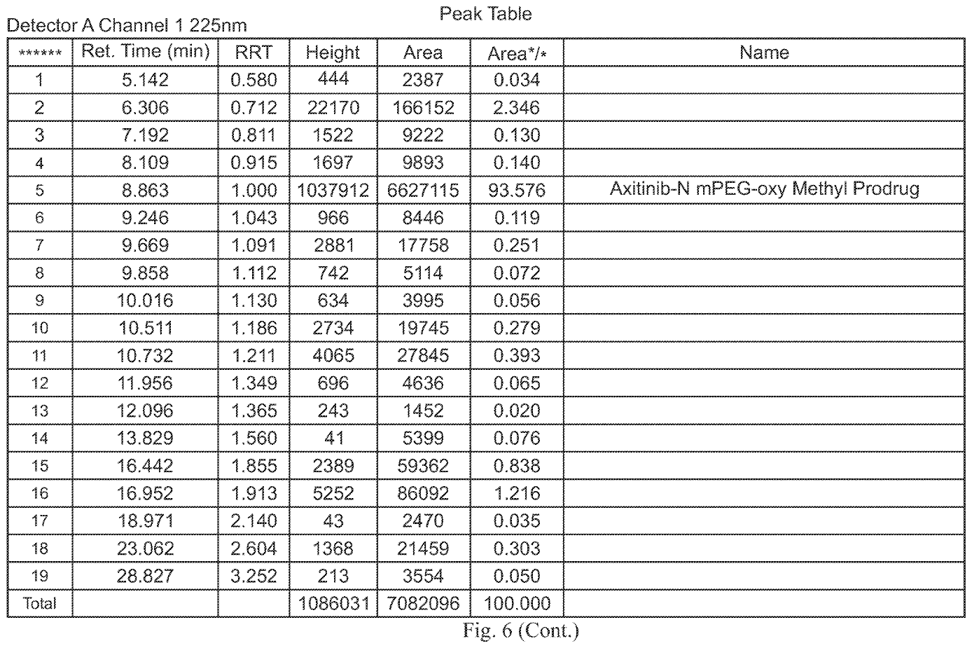

### DETAILED DESCRIPTION

In certain embodiments, the present invention is directed to a compound that is more hydrophilic than the compound of formula II that is convertible in-vivo to the compound of formula II.

In certain embodiments, the compound of formula I is converted in-vivo to a compound of formula II (axitinib)

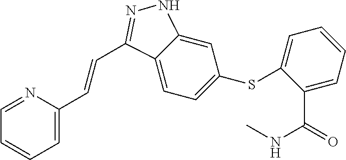 [SMILES: CNC(=O)c1ccccc1Sc1ccc2c(/C=C/c3ccccn3)n[nH]c2c1]

In certain embodiments, the present invention is directed to a compound of formula I:

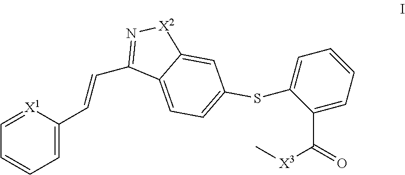

wherein:
X¹ is selected from N or N⁺Y¹;
X² is selected from NH or NY²;
X³ is selected from NH or NY³;
Y¹ is selected from —CH₂OCO(OCH₂CHO)n¹OM¹; or —CH₂OCO(CH₂CH₂O)n^{1a}Z¹; or —CH₂OCO(CH))n^{1b}COOH;
Y² is selected from —CH₂OCO(OCH₂CH₂)n²OM²; or —CH₂OCO(CH₂CH₂O)n^{2a}Z²; or —CH₂OCO(CH)n^{2b}COOH;
Y³ is selected from —CH₂OCO(OCH₂CH₂)n³OM³; or —CH₂OCO(CH₂CH₂O)n^{3a}Z³; or —CH₂OCO(CH₂)n^{3b}COOH;
n¹, n^{1a}, n^{1b}, n², n^{2a}, n^{2b}, n³, n^{3a} and n^{3b} are independently 0 or an integer from 1 to 8;
M¹, M², M³, Z¹, Z² and Z³ are independently selected from H, optionally substituted C₁₋₆alkyl and optionally substituted aryl;
wherein at least one of X¹, X² and X³ is not N or NH;
wherein at least one of Y¹, Y² or Y³ are the respective —CH₂OCO(CH₂CH₂O)nZ;
and pharmaceutically acceptable salts thereof.

In certain embodiments, the present invention is directed to a compound of formula I, wherein;
X¹ is NYE
X² is NH; and
X³ NH; and
Y¹ is —CH₂OCO(CH₂CH₂O)n¹Z¹;

In certain embodiments, the present invention is directed to a compound of formula I, wherein:

X¹ is N;
X² is NY³;
X³ is NH; and
Y² is —CH₂OCO(CH₂CH₂O)n²Z².

In certain embodiments, the present invention is directed to a compound of formula I, wherein:
X¹ is N;
X² is NH;
X³ is NY³, and
Y is —CH₂OCO(CH₂CH₂O)n³Z³.

In certain embodiments, the present invention is directed to a compound of formula I, wherein n¹ is 0, 1, 2, 3, 4, 5, 6, 7 or 8; or 1-3 or 4-6 or 7-8.

In certain embodiments, the present invention is directed to a compound of formula I, wherein n² is 0, 1, 2, 3, 4, 5, 6, 7 or 8; or 1-3 or 4-6 or 7-8.

In certain embodiments, the present invention is directed to a compound of formula I, wherein n³ is 0, 1, 2, 3, 4, 5, 6, 7 or 8; or 1-3 or 4-6 or 7-8.

In certain embodiments, the present invention is directed to a compound of formula I, wherein n^{1a} is 0, 1, 2, 3, 4, 5, 6, 7 or 8; or 1-3 or 4-6 or 7-8.

In certain embodiments, the present invention is directed to a compound of formula I, wherein n^{2a} is 0, 1, 2, 3, 4, 5, 6, 7 or 8; or 1-3 or 4-6 or 7-8.

In certain embodiments, the present invention is directed to a compound of formula I, wherein n^{3b} is 0, 1, 2, 3, 4, 5, 6, 7 or 8; or 1-3 or 4-6 or 7-8.

In certain embodiments, the present invention is directed to a compound of formula I, wherein n^{1b} is 0, 1, 2, 3, 4, 5, 6, 7 or 8; or 1-3 or 4-6 or 7-8.

In certain embodiments, the present invention is directed to a compound of formula I, wherein n^{2b} is 0, 1, 2, 3, 4, 5, 6, 7 or 8; or 1-3 or 4-6 or 7-8.

In certain embodiments, the present invention is directed to a compound of formula I, wherein n^{3b} is 0, 1, 2, 3, 4, 5, 6, 7 or 8; or 1-3 or 4-6 or 7-8.

In certain embodiments, M¹ is methyl, ethyl, propyl or phenyl.

In certain embodiments, M² is methyl, ethyl, propyl or phenyl.

In certain embodiments, M³ is methyl, ethyl, propyl or phenyl.

In certain embodiments, Z¹ is methyl, ethyl, propyl or phenyl.

In certain embodiments, Z² is methyl, ethyl, propyl or phenyl.

In certain embodiments, Z³ is methyl, ethyl, propyl or phenyl.

In certain embodiments, the compound of formula I is axitinib-N-mPEG-succinoyloxymethyl; or a pharmaceutically acceptable salt thereof.

In certain embodiments, the present invention is directed to a pharmaceutical composition comprising a compound of formula I as disclosed herein and a pharmaceutically acceptable excipient.

In certain embodiments, the pharmaceutical composition is in the form of an oral solid dosage form such as a tablet, a capsule or a powder.

In certain embodiments, the pharmaceutical composition is in the form of an ocular formulation such as an implant, an injection, a solution, a suspension or an ointment. The formulation can be administered intravitreally, topically or to any anterior or posterior section of the eye of a mammal (e.g., human).

In certain embodiments, a prodrug is included in a hydrogel (e.g., a polyethylene glycol based system as disclosed herein) or organogel, e.g., for ocular administration.

The prodrug included in the hydrogel or organogel can be a prodrug of axitinib as disclosed herein or any other prodrug of axitinib or prodrug of a different active agent known in the art. In certain embodiments, the selection of the hydrogel or organogel and the prodrug can be selected to manipulate the release of active agent from the dosage form. In such embodiments, the prodrug can increase or decrease the solubility of the base drug.

In certain embodiments, the prodrug included in a hydrogel or organogel can be as described in US2021/0078970, hereby incorporated by reference. These prodrugs include compounds of Formula I below, or pharmaceutically acceptable salts, esters, solvates, or polymorphs thereof:

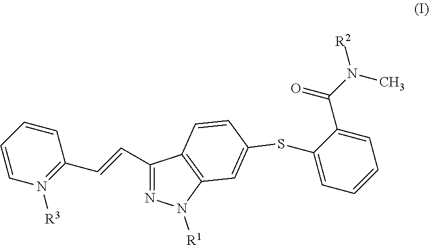

where R¹ and R² are independently a hydrogen (H) or a protecting group (P); R³, which may be present or absent, is a protecting group, and when R³ is present, the nitrogen atom is positively charged and a counterion is also present; provided that the compound of Formula I is not axitinib. In embodiments where both R¹ and R² are a protecting group (P), the protecting groups may be the same or different.

In one embodiment, a compound of Formula I is a compound of Formula II below, or a pharmaceutically acceptable salt, ester, solvate, or polymorph thereof:

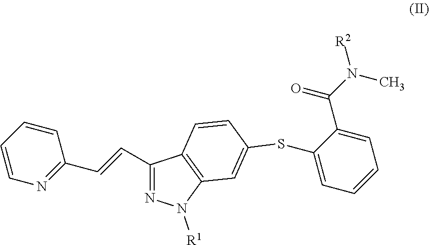

where R¹ and R² are independently a hydrogen (H) or a protecting group (P), and when both R¹ and R² are a protecting group, the protecting groups may be the same or different.

In another embodiment, a compound of Formula I is a compound of Formula III below, or a pharmaceutically acceptable salt, ester, solvate, or polymorph thereof.

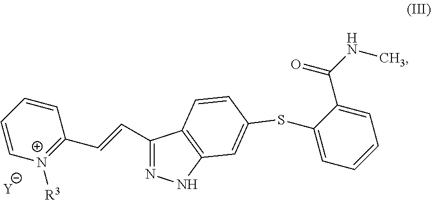

where R³ is a protecting group (P), and is a counterion.

In one embodiment, a protecting group is selected from acyl group, alkylcarbonyl group, arylcarbonyl group, alkylthiocarbonyl group, arylthiocarbonyl group, alkylcarbamoyl group, arylcarbamoyl group, substituted or unsubstituted acetyl, substituted or unsubstituted aminoalkanoyl, substituted or unsubstituted-aminoalkanoyl, acyl group derived from a natural or an unnatural amino acid with or without substitution, acyl group of a peptide residue, phosphonyl, phosphinyl, aminophophinyl, alkylaminophophinyl, sulfonyl, cycloalkane-carbonyl, heterocycloalkane-carbonyl, alkoxycarbonyl, aryloxycarbonyl, heteroalkoxycarbonyl, heteroaryloxycarbonyl, and O-substituted hydroxymethyl group with or without substituents.

In another embodiment, a protecting group is selected from R⁴W(R⁵R⁶C)ₘ—, where: m is an integer selected from 0 to 6; W is oxygen (—O—), sulfur (—S—), nitrogen (—NH—), or absent; R⁵ and R⁶ are independently a hydrogen or a lower alkyl group; and R⁴ is

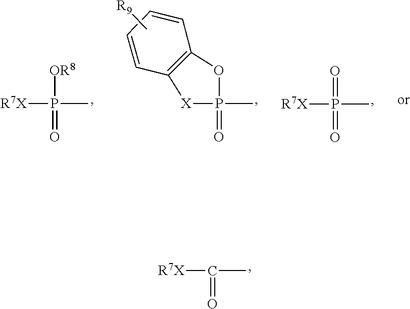

where X is oxygen (—O—), sulfur (—S—), nitrogen (NH—), or a methylene (—CH₂—) group, R⁶ and R⁷ are independently a hydrogen, a substituted or unsubstituted alkyl or cycloalkyl, an aryl or heteroaryl group without or with substitution, a PEG moiety (such as R¹⁰—(OCH₂CH)ₙ—, where n=1 to 10 and R¹⁰ is a hydrogen or a lower alkyl), an ester-forming group such as a lower alkyl or an aryl group; or a salt-forming moiety when X is oxygen or sulfur, such as a sodium, a potassium, a tetraethylammonium, or a tetrabutylammonium; or, the combination of R⁶ and X is an alky or aryl group with or without further substitution; provided that the compound of Formula I, II, or III is not axitinib.

In some embodiments, a counterion is selected from, but not limited to, halide ion (F⁻, Cl⁻, Br⁻, and I⁻), sulfate ion, methanesulfonate ion, toluenesulfonate ion, oxalate ion, and other pharmaceutically accepted anionic moiety.

In other embodiments, the prodrug included in a hydrogel or organogel can be as described in US20180022734, hereby incorporated by reference. These prodrugs include sunitinib compounds of Formula I below.

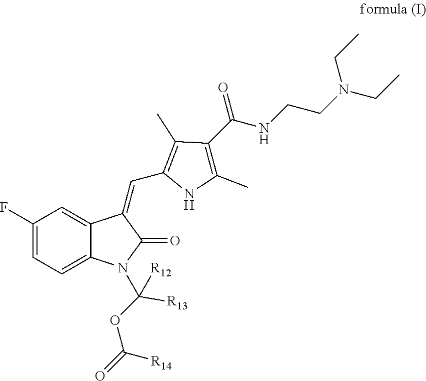

wherein:
R¹², R¹³ are selected from the group consisting of H, C₁-C₆ alkyl, C₂-C₆ alkenyl, C₂-C₆ alkynyl, C₃-C₈ cycloalkyl, C₆-C₁₀ aryl, 4-15 membered heterocyclyl and 5-15 membered heteroaryl;
R¹⁴ is selected from the group consisting of R′, OR′, SR′ and N(R′)₂; and
R′ is selected from the group consisting of H, C₁-C₆ alkyl, C₂-C₆ alkenyl, C₂-C₆ alkynyl, C₃-C₈ cycloalkyl, C₆-C₁₀ aryl, 4-15 membered heterocyclyl, 5-15 membered heteroaryl, hydroxyl C₁-C₆ alkyl, carboxyl C₁-C₆ alkyl, C₁-C₆ alkyl amido and phosphate group.

In some embodiments, R₁₂, R₁₃ are H, and R₁₄ is selected from the group consisting of N,N-dimethylaminomethyl, t-butyl, phenyl, p-fluorophenyl, biphenyl, dimethylamino, cyclopropyl, methyl, ethyl, propyl, isopropyl, butyl, isobutyl, cyclobutyl, pentyl, isopentyl, neopentyl, cyclopentyl, 1-methylcyclobutyl, N-methylamino, N-ethylamino, N,N-methylethylamino, n-hexyl, cyclohexyl, methylthio, ethylthio, propylthio, isopropylthio, cyclopropylthio, butylthio, isobutylthio, cyclobutylthio, methoxy, ethoxy, propoxy, isopropoxy, butoxy, cyclobutoxy, pentylamino, pentylthio, pentyloxy, isopentylamino, neopentylamino, t-butylamino, cyclopentylamino, cyclopentylthio, cyclopentyloxy, cyclohexylamino, cyclohexylthio, cyclohexyloxy, p-methoxyphenyl, p-chlorophenyl and o-fluorophenyl.

In certain embodiments, the increase in solubility of the axitinib prodrug allows for the adjustment of the release of the active from the hydrogel as compared to the base drug (i.e., axitinib). For example, the release rate can be at least 1.1 times higher, at least 1.2 times higher, at least 1.5 times higher, at least 2 times higher, at least 5 times higher, at least 10 times higher, at least 20 times higher, at least 50 times higher, at least 100 times higher, at least 250 times higher, at least 500 times higher or at least 1000 times higher, including all ranges between any of the previous values. In certain embodiments, the present invention is directed to a method of treating a disease or condition comprising administering a compound of formula I or pharmaceutical composition as disclosed herein.

In certain embodiments, the disease or condition is a cancer such as advanced renal cell carcinoma.

In certain embodiments, the disease or condition is an ocular disease or condition such as AMD, DME or RVO.

In certain embodiments, after administration to a patient or subject the compound of formula I is converted in-vivo to a compound of formula II

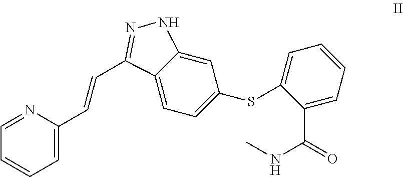 [SMILES: CNC(=O)c1ccccc1Sc1ccc2c(/C=C/c3ccccn3)n[nH]c2c1]

In certain embodiments, the present invention is directed to a method of treating a disease or condition by axitinib therapy comprising administering a compound or pharmaceutical composition as disclosed herein.

In certain embodiments, the invention is directed to a hydrogel comprising a compound as disclosed herein.

In certain embodiments, the invention is directed to a xerogel comprising a compound as disclosed herein.

In certain embodiments, the hydrogel or xerogel may be formed from precursors having functional groups that form crosslinks to create a polymer network. These crosslinks between polymer strands or arms may be chemical (i.e., may be covalent bonds) and/or physical (such as ionic bonds, hydrophobic association, hydrogen bridges etc.) in nature.

The polymer network may be prepared from precursors, either from one type of precursor or from two or more types of precursors that are allowed to react. Precursors are chosen in consideration of the properties that are desired for the resultant hydrogel. There are various suitable precursors for use in making the hydrogels and xerogels. Generally, any pharmaceutically acceptable and crosslinkable polymers forming a hydrogel may be used for the purposes of the present invention. The hydrogel and thus the components incorporated into it, including the polymers used for making the polymer network, should be physiologically safe such that they do not elicit e.g. an immune response or other adverse effects. Hydrogels and xerogels may be formed from natural, synthetic, or biosynthetic polymers. Natural polymers may include glycosaminoglycans, polysaccharides (e.g. dextran), polyaminoacids and proteins or mixtures or combinations thereof.

Synthetic polymers may generally be any polymers that are synthetically produced from a variety of feedstocks by different types of polymerization, including free radical polymerization, anionic or cationic polymerization, chain-growth or addition polymerization, condensation polymerization, ring-opening polymerization etc. The polymerization may be initiated by certain initiators, by light and/or heat, and may be mediated by catalysts.

Generally, for the purposes of the present invention one or more synthetic polymers of the group comprising one or more units of polyalkylene glycol, such as polyethylene glycol (PEG), polypropylene glycol, poly(ethylene glycol)-block-poly(propylene glycol) copolymers, or polyethylene oxide, polypropylene oxide, polyvinyl alcohol, poly (vinylpyrrolidinone), polylactic acid, polylactic-co-glycolic acid, random or block copolymers or combinations/mixtures of any of these can be used, while this list is not intended to be limiting.

To form covalently crosslinked polymer networks, the precursors may be covalently crosslinked with each other. In certain embodiments, precursors with at least two reactive centers (for example, in free radical polymerization) can serve as crosslinkers since each reactive group can participate in the formation of a different growing polymer chain.

The precursors may have biologically inert and hydrophilic portions, e.g., a core. In the case of a branched polymer, a core refers to a contiguous portion of a molecule joined to arms that extend from the core, where the arms carry a functional group, which is often at the terminus of the arm or branch. Multi-armed PEG precursors are examples of such precursors and are further disclosed herein below.

Thus a hydrogel for use in the present invention can be made e.g. from one multi-armed precursor with a first (set of) functional group(s) and another multi-armed precursor having a second (set of) functional group(s). By way of example, a multi-armed precursor may have hydrophilic arms, e.g., polyethylene glycol units, terminated with primary amines (nucleophile), or may have activated ester end groups (electrophile). The polymer network according to the present invention may contain identical or different polymer units crosslinked with each other.

Certain functional groups can be made more reactive by using an activating group. Such activating groups include (but are not limited to) carbonyldiimidazole, sulfonyl chloride, aryl halides, sulfosuccinimidyl esters, N-hydroxysuccinimidyl ester, succinimidyl ester, epoxide, aldehyde, maleimides, imidoesters, acrylates and the like. The N-hydroxysuccinimide esters (NHS) are useful groups for crosslinking of nucleophilic polymers, e.g., primary amine-terminated or thiol-terminated polyethylene glycols. An NHS-amine crosslinking reaction may be carried out in aqueous solution and in the presence of buffers, e.g., phosphate buffer (pH 5.0-7.5), triethanolamine buffer (pH 7.5-9.0), borate buffer (pH 9.0-12), or sodium bicarbonate buffer (pH 9.0-10.0).

In certain embodiments, each precursor may comprise only nucleophilic or only electrophilic functional groups, so long as both nucleophilic and electrophilic precursors are used in the crosslinking reaction. Thus, for example, if a crosslinker has only nucleophilic functional groups such as amines, the precursor polymer may have electrophilic functional groups such as N-hydroxysuccinimides. On the other hand, if a crosslinker has electrophilic functional groups such as sulfosuccinimides, then the functional polymer may have nucleophilic functional groups such as amines or thiols. Thus, functional polymers such as proteins, poly (allyl amine), or amine-terminated di-or multifunctional poly(ethylene glycol) can be also used to prepare the polymer network of the present invention.

In one embodiment a first reactive precursor has about 2 to about 16 nucleophilic functional groups each (termed functionality), and a second reactive precursor allowed to react with the first reactive precursor to form the polymer network has about 2 to about 16 electrophilic functional groups each. Reactive precursors having a number of reactive (nucleophilic or electrophilic) groups as a multiple of 4, thus for example 4, 8 and 16 reactive groups, are particularly suitable for the present invention. Any number of functional groups, such as including any of 2, 3, 4, 5, 6, 7, 8, 9, 10, 11, 12, 13, 14, 15, or 16 groups, is possible for precursors to be used in accordance with the present invention, while ensuring that the functionality is sufficient to form an adequately crosslinked network.

In a certain embodiments of the present invention, the polymer network forming the hydrogel contains polyethylene glycol (PEG) units. PEGs are known in the art to form hydrogels when crosslinked, and these PEG hydrogels are suitable for pharmaceutical applications e.g. as matrix for drugs intended to be administered to all parts of the human or animal body.

The polymer network of the hydrogel implants of the present invention may comprise one or more multi-arm PEG units having from 2 to 10 arms, or 4 to 8 arms, or 4, 5, 6, 7 or 8 arms. The PEG units may have a different or the same number of arms. In certain embodiments, the PEG units used in the hydrogel of the present invention have 4 and/or 8 arms. In certain particular embodiments, a combination of 4- and 8-arm PEG units is utilized.

The number of arms of the PEG used contributes to controlling the flexibility or softness of the resulting hydrogel. For example, hydrogels formed by crosslinking 4-arm PEGs are generally softer and more flexible than those formed from 8-arm PEGs of the same molecular weight. In particular, if stretching the hydrogel prior to or after drying as disclosed herein below in the section relating to the manufacture of the implant is desired, a more flexible hydrogel may be used, such as a 4-arm PEG, optionally in combination with another multi-arm PEG, such as an 8-arm PEG as disclosed above.

In certain embodiments of the present invention, polyethylene glycol units used as precursors have an average molecular weight in the range from about 2,000 to about 100,000 Daltons, or in a range from about 10,000 to about 60,000 Daltons, or in a range from about 15,000 to about 50,000 Daltons. In certain particular embodiments the polyethylene glycol units have an average molecular weight in a range from about 10,000 to about 40,000 Daltons, or of about 20,000 Daltons. PEG precursors of the same average molecular weight may be used, or PEG precursors of different average molecular weight may be combined with each other. The average molecular weight of the PEG precursors used in the present invention is given as the number average molecular weight (Mn), which, in certain embodiments, may be determined by MALDI.

In a 4-arm PEG, each of the arms may have an average arm length (or molecular weight) of the total molecular weight of the PEG divided by 4. A 4a20kPEG precursor, which is one precursor that can be utilized in the present invention thus has 4 arms with an average molecular weight of about 5,000 Daltons each. An 8a20k PEG precursor, which may be used in addition to the 4a20kPEG precursor in the present invention, thus has 8 arms each having an average molecular weight of 2,500 Daltons. Longer arms may provide increased flexibility as compared to shorter arms. PEGs with longer arms may swell more as compared to PEGS with shorter arms. A PEG with a lower number of arms also may swell more and may be more flexible than a PEG with a higher number of arms. In certain particular embodiments, combinations of PEG precursors with different numbers of arms, such as a combination of a 4-arm PEG precursor and an 8-arm precursor, may be utilized in the present invention. In addition, longer PEG arms have higher melting temperatures when dry, which may provide more dimensional stability during storage. For example, an 8-arm PEG with a molecular weight of 15,000 Dalton crosslinked with trilysine may not be able to maintain a stretched configuration at room temperature, whereas a 4-arm 20,000 Dalton PEG crosslinked with an 8-arm 20,000 Dalton PEG may be dimensionally stable in a stretched configuration at room temperature.

When referring to a PEG precursor having a certain average molecular weight, such as a 15kPEG- or a 20kPEG-precursor, the indicated average molecular weight (i.e., a Mn of 15,000 or 20,000, respectively) refers to the PEG part of the precursor, before end groups are added (“20k” here means 20,000 Daltons, and “15k” means 15,000 Daltons—the same abbreviation is used herein for other average molecular weights of PEG precursors). In certain embodiments, the Mn of the PEG part of the precursor is determined by MALDI. The degree of substitution with end groups as disclosed herein may be determined by means of ¹H-NMR after end group functionalization.

In certain embodiments, electrophilic end groups for use with PEG precursors for preparing the hydrogels of the present invention are N-hydroxysuccinimidyl (NHS) esters, including but not limited to: “SAZ” referring to a succinimidylazelate end group, “SAP” referring to a succinimidyladipate end group, “SG” referring to a succinimidylglutarate end group, and “SS” referring to a succinimidylsuccinate end group.

In certain embodiments, nucleophilic end groups for use with PEG precursors for preparing the hydrogels of the present invention are amine (denoted as “NH₂”) end groups. Thiol (—SH) end groups or other nucleophilic end groups are also possible.

In certain preferred embodiments, 4-arm PEGs with an average molecular weight of about 20,000 Daltons and an electrophilic end group as disclosed above and 8-arm PEGs also with an average molecular weight of about 20,000 Daltons and with a nucleophilic end group as disclosed above are crosslinked for forming the polymer network and thus the hydrogel according to the present invention.

Reaction of nucleophilic group-containing PEG units and electrophilic group-containing PEG units, such as amine end-group containing PEG units and activated ester-group containing PEG units, results in a plurality of PEG units being crosslinked by a hydrolyzable linker having the formula:

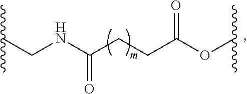

wherein m is an integer from 0 to 10, and specifically is 1, 2, 3, 4, 5, 6, 7, 8, 9, or 10. In one particular embodiment, m is 6, e.g. in the case a SAZ-end group-containing PEG is used. For a SAP-end group, m would be 3, for a SG-end group, m would be 2 and for an SS-end group m would be 1. All crosslinks within the polymer network may be the same, or may be different.

In certain preferred embodiments, the SAZ end group is utilized in the present invention. This end group may provide for increased duration in the eye, and the implant of certain embodiments of the present invention comprising a hydrogel comprising PEG-SAZ units is biodegraded in the eye, such as in the vitreous humor of a human eye, only after an extended period of time, e.g., 9 to 12 months as further disclosed below, and may in certain circumstance persist even longer. The SAZ group is more hydrophobic than e.g. the SAP-SG- or SS-end groups because of a higher number of carbon atoms in the chain (m being 6, and the total of carbon atoms between the amide group and the ester group being 7).

In certain preferred embodiments, a 4-arm 20,000 Dalton PEG precursor is combined with an 8-arm 20,000 Dalton PEG precursor, such as a 4-arm 20,000 Dalton PEG precursor having a SAZ group (as defined above) combined with an 8-arm 20,000 Dalton PEG precursor having an amine group (as defined above). These precursors are also abbreviated herein as 4a20kPEG-SAZ and 8a20kPEG-NH₂, respectively. The chemical structure of 4a20kPEG-SAZ is:

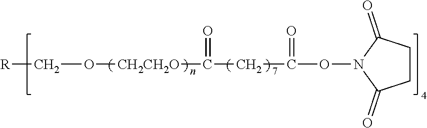

wherein R represents a pentaerythritol core structure. The chemical structure of 8a20kPEG₂-NH, (with a hexaglycerol core) is:

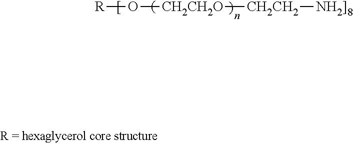

In the above formulae, n is determined by the molecular weight of the respective PEG-arm.

In certain embodiments, the molar ratio of the nucleophilic and the electrophilic end groups reacting with each other is about 1:1, i.e., one amine group is provided per one SAZ group. In the case of 4a20kPEG-SAZ and 8a20kPEG-NH₂ this results in a weight ratio of about 2:1, as the 8-arm PEG contains double the amount of end groups as the 4-arm PEG. However, an excess of either the electrophilic (e.g. the NHS end groups, such as the SAZ) end groups or of the nucleophilic (e.g. the amine) end groups may be used. In particular, an excess of the nucleophilic, such as the amine-end group containing precursor may be used, i.e., the weight ratio of 4a20kPEG-SAZ and 8a20kPEG-NH₂ may also be less than 2:1.

Each and any combination of electrophilic- and nucleophilic-group containing PEG precursors disclosed herein may be used for preparing the implant according to the present invention. For example, any 4-arm or 8-arm PEG-NHS precursor (e.g. having a SAZ, SAP, SG or SS end group) may be combined with any 4-arm or 8-arm PEG-NH₂ precursor (or any other PEG precursor having a nucleophilic group). Furthermore, the PEG units of the electrophilic- and the nucleophilic group-containing precursors may have the same, or may have a different average molecular weight.

Another nucleophilic group-containing crosslinking agent may be used instead of a PEG-based crosslinking agent. For example, a low-molecular weight amine linker can be used, such as trilysine (or a trilysine salt or derivative, such as trilysine acetate) or other low-molecular weight multi-arm amines.

In certain embodiments, the nucleophilic group-containing crosslinking agent may be bound to or conjugated with a visualization agent. A visualization agent is an agent that contains a fluorophoric or other visualization-enabling group. Fluorophores such as fluorescein, rhodamine, coumarin, and cyanine may for example be used as visualization agents. The visualization agent may be conjugated with the crosslinking agent e.g. through some of the nucleophilic groups of the crosslinking agent. Since a sufficient amount of the nucleophilic groups are necessary for crosslinking, “conjugated” or “conjugation” in general includes partial conjugation, meaning that only a part of the nucleophilic groups are used for conjugation with the visualization agent, such as about 1% to about 20%, or about 5% to about 10%, or about 8% of the nucleophilic groups of the crosslinking agent may be conjugated with a visualization agent. In other embodiments, a visualization agent may also be conjugated with the polymer precursor, e.g. through certain reactive (such as electrophilic) groups of the polymer precursors.

The disclosures herein for hydrogels can also be applicable to xerogels.

### Extruded Dosage Forms

The materials disclosed herein for hydrogels can also be extruded with a prodrug.

In certain embodiments is directed to a method of preparing a sustained release biodegradable ocular insert comprising extruding a polymer composition and a prodrug to form an insert suitable for ocular administration.

In other embodiments, the method comprises feeding the polymer composition and the prodrug into an extruder; mixing the components in the extruder; extruding a strand; and cutting the strand into unit dose inserts or implants.

In certain embodiments, the polymer composition and the prodrug are fed separately into the extruder. In other embodiments, the polymer composition and prodrug are fed simultaneously into the extruder. In certain embodiments, the polymer composition are pre-mixed, e.g., melt blended, prior to introduction into the extruder.

In certain embodiments, the method further comprising cooling the strand, e.g., prior to cutting the strand.

In certain embodiments, the method further comprises stretching the strand, e.g., prior to cutting the strand.

In certain embodiments, the stretching is performed under wet conditions, heated conditions, or a combination thereof. In other embodiments, the stretching is performed under dry conditions, heated conditions, or a combination thereof.

In certain embodiments, the extruded composition is subject to a curing step, e.g., humidity exposure. In certain embodiments, the curing crosslinks the polymer composition.

In certain embodiments, the method further comprises drying the strand after stretching the strand.

In other embodiments, any of the method steps disclosed herein can be performed simultaneously or sequentially in any order.

In certain embodiments, the method further comprises melting the polymer in the extruder at a temperature below the melting point of the prodrug. The temperature can be, e.g., less than about 100°, less than about 90°, less than about 80°, less than about 70°, less than about 60°, less than about 50°. In some embodiments, the temperature is from about 50° to about 80 °C. In certain embodiments, the extrusion is performed above the melting point of the polymer and the prodrug.

In certain embodiments, the extruded composition is dried, when in strand form or in unit doses. In certain embodiments, the drying is performed after stretching the strand. The drying can be, e.g., evaporative drying at ambient temperatures or can include heat, vacuum or a combination thereof.

In certain embodiments, the hydrogel strand is stretched by a stretch factor in the range of about 0.25 to about 10, 0.5 to about 6 or about 1 to about 4.

In certain embodiments, the strand is cut into segments having an average length of equal to or less than about 20 mm, 17 mm, 15 mm, 12 mm, 10 mm, 8 mm, 5 mm, 4 mm, 3 mm, 2 mm, 1 mm or 0.5 mm.

In certain embodiments, the prodrug is suspended in the polymer composition.

In certain embodiments, the prodrug is homogenousely dispersed in the polymer composition.

In certain embodiments, the extrusion process is performed without solvent (e.g., water), In certain embodiments a solvent is used in an amount of less than about 10% w/w, less than about 5% w/w or less than about 1% w/w.

In certain embodiments, the content uniformity of the unit dose insert is within 10%, with 5% or within 1%.

In certain embodiments, the persistence of the dosage form is from about 7 days to about 6 months after ocular administration.

In certain embodiments, the polymorphic form of the prodrug does not change or does not substantially change. In certain embodiments, the purity of the prodrug after curing is greater than 99%, greater than 99.5% or greater than 99.9% as compared to the prodrug prior to extrusion.

In certain embodiments, the prodrug has an average particle diameter of less than about 100 μm, less than about 50 μm, less than about 25 μm, or less than about 10 μm.

In certain embodiments, the prodrug has a D50 particle size of less than about 10 μm and/or a D99 particle size of less than about 50 μm, or a D90 particle size of about 5 μm or less and/or a D98 particle size of about 10 μm or less.

### Organogels

In certain embodiments, the prodrugs herein can be used in an organogel.

In certain embodiments, the present invention provides a sustained release, biodegradable drug-delivery system, comprising an organogel and a prodrug, the organogel comprising a hydrophobic organic liquid, and a biodegradable, covalently crosslinked polymeric network, wherein the hydrophobic organic liquid and the prodrug are contained in the biodegradable, covalently crosslinked polymeric network. In certain embodiments, a sustained release, biodegradable drug-delivery system is provided, comprising an organogel and a prodrug, the organogel comprising a hydrophobic organic liquid, and a biodegradable, covalently crosslinked polymeric network, wherein the hydrophobic organic liquid and the prodrug are immobilized in the biodegradable, covalently crosslinked polymeric network.

The sustained release, biodegradable drug-delivery system in certain embodiments comprises at least three constituents, a biodegradable covalently crosslinked polymer network, a hydrophobic organic liquid and a prodrug.

The organogel in certain embodiments is formed by polymerization of non-linear, multifunctional monomeric or polymeric precursor components as disclosed herein later and forms a covalently crosslinked polymeric network that includes the hydrophobic organic liquid and immobilizes it within the polymeric network, e.g., until it is released from the network in vivo. The organogels of the present invention are thus like a hydrophobic analog to hydrogels that include water instead of a hydrophobic organic phase. Organogels are similar to hydrogels in that the matrix is composed of a network forming polymeric component (gelator) and a non-reactive component. In a hydrogel the non-reactive component is water, whereas in the organogel of the present invention it is a hydrophobic organic compound with glass (Tg) and melt (Tm) transition temperatures below body temperature, such as an oil.

Covalent crosslinking of the polymer network forming precursors in certain embodiments provides a limited mobility to the hydrophobic organic liquid (e.g., oil) component. This may provide continuous control of drug release by limiting drug transport to diffusion through the organogel and/or eliminating development of defects that provide fast escape routes for the drug from developing. In certain embodiments, the drug-delivery system of the present invention is a fully or partly diffusion controlled delivery system, i.e. the release of the oil and/or the prodrug is primarily controlled by diffusion processes. Degradation of the polymer matrix may additionally occur in the organogels of the present invention, but does not primarily control the release of the prodrug. In non-crosslinked gels such as extruded linear polymers the release of the prodrug is mainly controlled by degradation of the polymeric matrix that releases the prodrug mainly in a degradation controlled system. The network forming precursors should be miscible in the hydrophobic organic liquid component, such that when crosslinked it “holds” the component to create a solid or semi-solid, that forms the organogel. In certain embodiments, the hydrophobic organic liquid compatibility with the polymer network has an impact on the rate the hydrophobic organic liquid escapes into the surrounding tissue fluid in vivo, and may gradually be replaced by aqueous fluid, providing an additional method of controlling drug release kinetics to prodrug solubility and network degradation.

In certain embodiments, the use of an organogel in the sustained release, biodegradable drug-delivery system of the invention thus allows to modify the release of a prodrug from the drug-delivery system by tailoring or suitably selecting the precursor components forming the crosslinked polymeric network according to their hydrophilic and/or hydrophobic properties. Furthermore, in certain embodiments, the release of a prodrug from the drug-delivery system can be modified or controlled by suitably selecting the hydrophobic organic liquid according to its properties such as hydrophobicity, viscosity, compatibility with the prodrug, solubility or insolubility of the prodrug in the hydrophobic organic phase, and the like.

The organogel based drug-delivery system of certain embodiments of the present invention offers several advantages over hydrogels. For example, certain organogels are anhydrous, so water degradable (hydrolysable) components such as water sensitive prodrugs can be stabilized and made storage stable over extended periods of time and don't require hydration at the time of implantation.

Water soluble compounds have low solubility or are insoluble in organogels, allowing the drug to be incorporated as a particulate solid embedded in the organogel matrix. The low solubility of the drug in the organogel matrix provides a mechanism to control the rate of drug release. This property vastly increases the range of compounds that can be contained in an implant.

Manipulation of the lipophilicity/hydrophilicity of the organogel can be used to adjust the release rate of a drug and to influence diffusion rates. Pure hydrogels cannot be adjusted this way, since they are water-based, thus in these systems the drug itself has to be modified to a prodrug form for such adjustment of drug/matrix solubility. In organogels, this can be avoided. Additionally, varying the lipophilicity/hydrophilicity of the organogel can be further used influence degradation rate of the polymer matrix, also having an additional influence on the release rate of a drug.

The organogel can be designed to release the hydrophobic organic liquid (e.g., oil) from the matrix slowly in vivo, allowing a slow conversion to hydrogel that is then degraded. This provides a new mode of drug release control and increases biocompatibility. Optional addition of a solvent in organogels can be used during manufacture to overcome compatibility issues of the components, and the solvent can be removed to yield an organogel with an immobilized oil. Removal of the solvent can be accomplished by heat treatment, which is not possible for materials that undergo melting or glass transitions at elevated temperatures. Solvent removal can also be accomplished by methods typically employed for non-crosslinked polymers, such as water extraction, vacuum drying, lyophilization, evaporation, etc. Elimination of the need for careful removal of solvent greatly simplifies the fabrication process.

In certain embodiments, organogels possess the physical qualities of low modulus, dimensional stability, and favorable drug release kinetics. In certain embodiments, organogels can be dimensionally stable to heat and will not melt. Thus, implant fabrication processes such as hot melt extrusion can be used to form certain organogels of the present invention.

The drug-delivery system of the present invention comprising an organogel may be used to deliver classes of drugs including steroids, non-steroidal anti-inflammatory drugs (NSAIDS), intraocular pressure lowering drugs, antibiotics, peptides, or others. The organogel may be used to deliver drugs and therapeutic agents, e.g., an anti-inflammatory (e.g., Diclofenac), a pain reliever (e.g., Bupivacaine), a calcium channel blocker (e.g., Nifedipine), an Antibiotic (e.g., Ciprofloxacin), a cell cycle inhibitor (e.g., Simvastatin), a protein or peptide (e.g., Insulin), enzymes, antineoplastic agents, local anesthetics, hormones, angiogenic agents, anti-angiogenic agents, growth factors, antibodies, neurotransmitters, psychoactive drugs, anticancer drugs, chemotherapeutic drugs, drugs affecting reproductive organs, genes, and oligonucleotides, or other configurations, as well as viruses such as AAV for gene delivery. The rate of release from the organogel may depend on the properties of one or more of the prodrug, the hydrophobic organic liquid and the polymer network, with other possible factors including one or more of drug sizes, relative hydrophobicity, organogel density, organogel solids content, and the like.

The drug-delivery system of the present invention may be in the form of an implant, a medical implant or a pharmaceutically acceptable implant, an implant coating, or an oral dosage form, etc.

### Methods of Treatment

In certain embodiments the prodrugs disclosed herein are utilized in the treatment of ocular disease involving angiogenesis.

In other embodiments the ocular disease may be mediated by one or more receptor tyrosine kinases (RTKs), such as VEGFR-1, VEGFR-2, VEGFR-3, PDGFR-α/β, and/or by c-Kit.

In some embodiments the ocular disease is a retinal disease including Choroidal Neovascularization, Diabetic Retinopathy, Diabetic Macula Edema, Retinal Vein Occlusion, Acute Macular Neuroretinopathy, Central Serous Chorioretinopathy, and Cystoid Macular Edema; wherein the ocular disease is Acute Multifocal Placoid Pigment Epitheliopathy, Behcet's Disease, Birdshot Retinochoroidopathy, Infectious (Syphilis, Lyme, Tuberculosis, Toxoplasmosis), Intermediate Uveitis (Pars Planitis), Multifocal Choroiditis, Multiple Evanescent White Dot Syndrome (MEWDS), Ocular Sarcoidosis, Posterior Scleritis, Serpignous Choroiditis, Subretinal Fibrosis, Uveitis Syndrome, or Vogt-Koyanagi-Harada Syndrome; wherein the ocular disease is a vascular disease or exudative diseases, including Coat's Disease, Parafoveal Telangiectasis, Papillophlebitis, Frosted Branch Angitis, Sickle Cell Retinopathy and other Hemoglobinopathies, Angioid Streaks, and Familial Exudative Vitreoretinopathy; or wherein the ocular disease results from trauma or surgery, including Sympathetic Ophthalmia, Uveitic Retinal Disease, Retinal Detachment, Trauma, Photodynamic Laser Treatment, Photocoagulation, Hypoperfusion During Surgery, Radiation Retinopathy, or Bone Marrow Transplant Retinopathy.

In alternative embodiments the prodrugs utilized herein can be utilized in the treatment of ocular conditions associated with tumors. Such conditions include e.g., Retinal Disease Associated with Tumors, Solid Tumors, Tumor Metastasis, Benign Tumors, for example, hemangiomas, neurofibromas, trachomas, and pyogenic granulomas, Congenital Hypertrophy of the RPE, Posterior Uveal Melanoma, Choroidal Hemangioma, Choroidal Osteoma, Choroidal Metastasis, Combined Hamartoma of the Retina and Retinal Pigmented Epithelium, Retinoblastoma, Vasoproliferative Tumors of the Ocular Fundus, Retinal Astrocytoma, or Intraocular Lymphoid Tumors.

In other embodiments, the prodrugs of the present invention can be utilized in the treatment of any ocular disease involving vascular leakage.

In certain embodiments the ocular disease is selected from neovascular age-related macular degeneration (AMD), diabetic macula edema (DME), and retinal vein occlusion (RVO). In particular embodiments the ocular disease is neovascular age-related macular degeneration. In other embodiments, the ocular condition is dry eye.

The compounds and pharmaceutical compositions disclosed herein can be administered by any route such as oral, parenteral, ocular, transdermal, nasal, pulmonary or rectal. In certain embodiments, the administration can be to a vitreous body, or other location. Examples of another space are puncta (canaliculus, upper/lower canaliculus), ocular fornix, upper/lower ocular fornix, subtenon space, choroid, suprachoroid, tenon, cornea, cancer tissue, organ, prostate, breast, surgically created space or injury, void space, and potential space. In certain embodiments, the dosage form is a punctal plug, intracanalicular insert, intracameral insert or intravitreal insert.

In particular embodiments, the prodrugs and formulations herein are used (e.g., for oncology treatment) by intravitreal, suprachoroidal, subretinal, subconjunctival or subtenon administration.

In certain embodiments, the invention is directed to a prodrug (e.g., a prodrug of axitinib as disclosed herein) administered in combination with the base drug (e.g., axitinib) in the same formulation (e.g., an ocular formulation) or a different formulation in order to provide a quicker releasing loading dose when there is a lag time in the release or therapeutic effect of the base drug. In embodiments wherein the prodrug has a slower release than the base drug, the combination can provide a longer duration of effect than administration of the base drug alone.

In certain embodiments, the axitinib prodrug can be formulated and./or admibnsisted in accordance with U.S. Pat. No. 11,439,592 B2.

### EXAMPLES

### Example 1

### axitinib-N-mPEG-succinoyloxymethyl prodrug

The compound of Example 2 was prepared according to the schemes below:

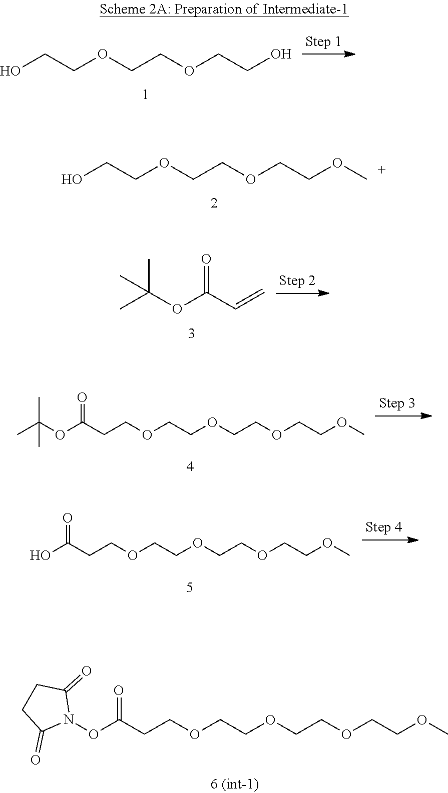

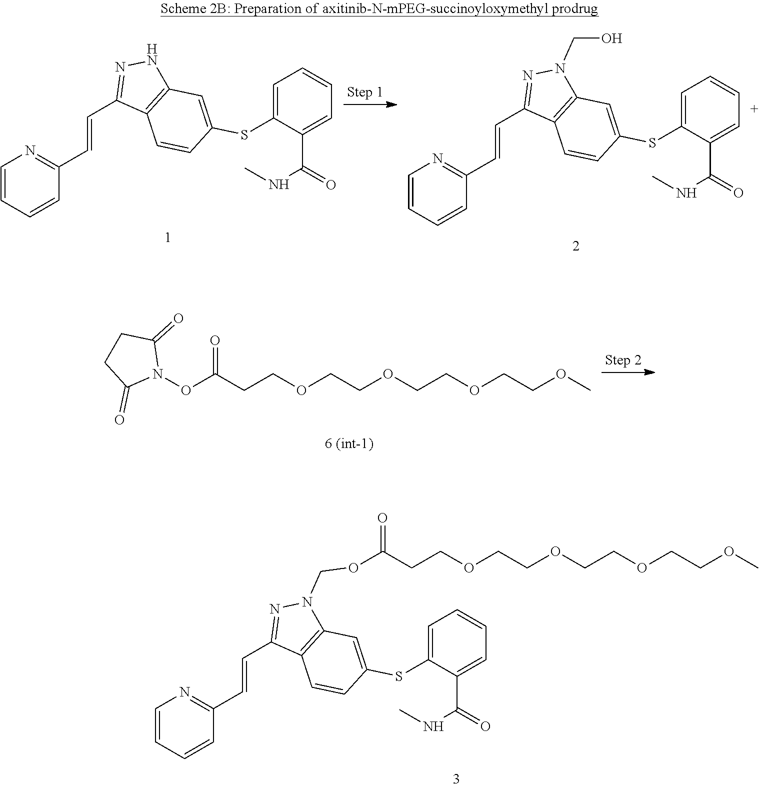 [SMILES: CNC(=O)c1ccccc1Sc1ccc2c(/C=C/c3ccccn3)n[nH]c2c1 ; CNC(=O)c1ccccc1Sc1ccc2c(/C=C/c3ccccn3)nn(CO)c2c1 ; COCCOCCOCCOCCC(=O)ON1C(=O)CCC1=O ; CNC(=O)c1ccccc1Sc1ccc2c(/C=C/c3ccccn3)nn(COC(=O)CCOCCOCCOCCOC)c2c1]

The sample number, batch size, conditions, yield and discussion are set forth below for each step of the process:

| | | | | |
|---|---|---|---|---|
| | Batch | | | |
| S. No | size | Conditions | Yield | Discussion |

Step-1

| | | | | |
|---|---|---|---|---|
| 1 | 2.0 g | Methyl iodide (0.5 eq.), | 400 mg | Formation of |
| | (1) | KOH (0.55 eq.), THF, | (20%) | compound 2 was |
| | | 0 °C-RT, 16 h | | confirmed by MS and |
| | | | | NMR. |
| | | | | Isolated compound 2 |
| | | | | by silica gel column |
| | | | | chromatography in |
| | | | | 400 mg scale. |

Step-2

| | | | | |
|---|---|---|---|---|
| 2 | 2.0 g | t-Butyl acrylate(3.0 | 150 g | Formation of |
| | (2) | eq.), Triton-B (40% in | (42%) | compound 4 was |
| | | methanol) (0.4 eq.), | | confirmed by NMR. |
| | | 0 °C-RT, 2 h. | | Isolated compound 4 |
| | | | | by silica gel column |
| | | | | chromatography in |
| | | | | 1.5 g scale. |

Step-3

| 2 | 1.5 g | Triflouroacetic acid | 850 mg | Formation of |
|---|---|---|---|---|
| | (4) | (5 V), DCM (5 V), | (70%) | compound 5 was |
| | | 0 °C-RT, 2 h | | confirmed by MS |
| | | | | and NMR. |
| | | | | Isolated compound 5 |
| | | | | by silica gel column |
| | | | | chromatography in |
| | | | | 850 mg scale. |
| | Batch | | | |
| S. No | size | Conditions | Yield | Comments |

Step-4

| | | | | |
|---|---|---|---|---|
| 1 | 0.1 g | N-Hydroxy succinimide | 110 mg | Attempt to convert |
| | (5) | (1.2 eq.), DIPEA (3.0 | (crude) | compound 5 to |
| | | eq), DCC (1.2 eq.), | | compound 6 was |
| | | DMAP (0.1 eq ), THF | | successful. |
| | | 0 °C-RT, 12 h | | Crude compound was |
| | | | | taken forward to next |
| | | | | step without |
| | | | | purification. |
| 2 | 0.1 g | DSC (1.2 eq.), DIPEA | — | Attempt to convert |
| | (5) | (2.0 eq), DMF | | compound 5 to |
| | | 45 °C, 16 h. | | compound 6 was not |
| | | | | successful. |
| | | | | SM intact by TLC. |
| 4 | 0.2 g | N-Hydroxy succinimide | 0.44 g | Formation of |
| | (5) | (1.1 eq.), EDC.HCl | (crude) | compound 6 was |
| | | (1.0 eq.), DCM, | | confirmed by NMR. |
| | | 0 °C-RT, 12 h | | Isolated compound 6 |
| | | | | (int-1) in 0.44 g scale. |

Step-2

| | | | | |
|---|---|---|---|---|
| 2 | 0.1 g | 6 (1.2 eq), DIPEA (2.0 | 30 mg | Formation of |
| | (2) | eq), DMF 0 °C-RT, | | compound 3 was |
| | | 16 h. | | confirmed by |
| | | | | LCMS (83.8%). |
| | | | | Isolated compound |
| | | | | 3 by silica gel |
| | | | | column |
| | | | | chromatography in |
| | | | | 30 mg scale. |
| 3 | 0.1 g | 6 (1.2 eq), TEA (2.0 | 120 mg | Formation of |
| | (2) | eq), DMF 0 °C-RT, | (16%) | compound 3 was |
| | | 16 h. | | confirmed by |
| | | | | LCMS and NMR. |
| | | | | Isolated compound |
| | | | | 3 by silica gel |
| 4 | 0.4 g | | | column |
| | (2) | | | chromatography in |
| | | | | 120 mg scale with |
| | | | | 95.53% LCMS |
| | | | | purity. |
| | | | | Purity by HPLC is |
| | | | | 93.57%. |
| | | | | Nature of the |
| | | | | compound: viscous |
| | | | | liquid. |
| | | | | Traces of DCM by |
| | | | | 1H-NMR were |
| | | | | observed and |
| | | | | further drying is in |
| | | | | order. |

### Scheme 1b (Alternate route): Preparation of axitinib-N-mPEG-succinoyloxymethyl prodrug

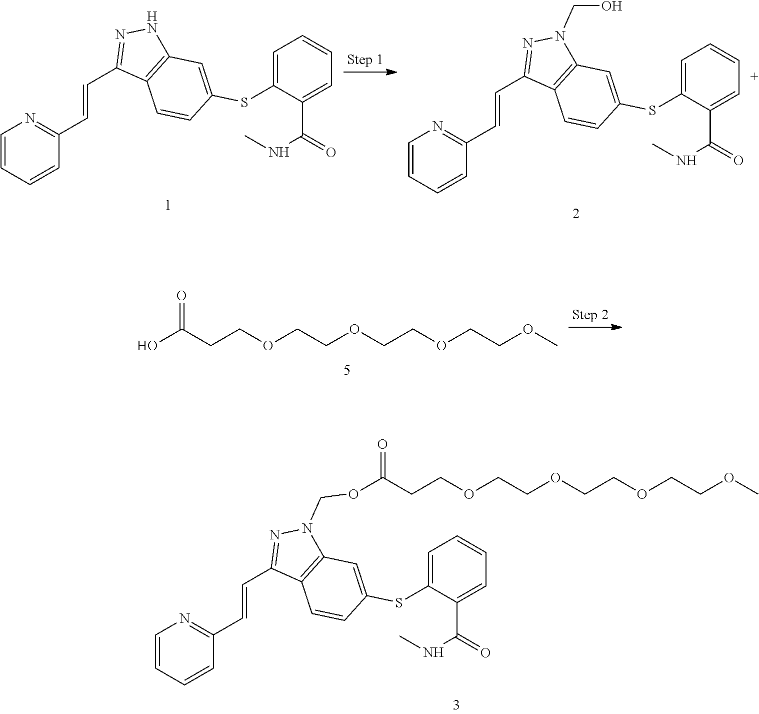 [SMILES: CNC(=O)c1ccccc1Sc1ccc2c(/C=C/c3ccccn3)n[nH]c2c1 ; CNC(=O)c1ccccc1Sc1ccc2c(/C=C/c3ccccn3)nn(CO)c2c1 ; COCCOCCOCCOCCC(=O)O ; CNC(=O)c1ccccc1Sc1ccc2c(/C=C/c3ccccn3)nn(COC(=O)CCOCCOCCOCCOC)c2c1]

| | | | | |
|---|---|---|---|---|
| S. No | Batch size | Conditions | Yield | Comments |

Step-2

| | | | | |
|---|---|---|---|---|
| 1 | 0.1 g | 5 (1.1 eq), EDC.HCl | 20 mg | Formation of |
| | (2) | (1.2 eq.), DIPEA | | compound 3 was |
| | | (2.0 eq), DMAP (0.1 | | confirmed by |
| | | eq.), DMF | | LCMS(83%) and |
| | | 0 °C-RT, 16 h. | | NMR. |
| | | | | Isolated compound 3 |
| | | | | by silica gel column |
| | | | | chromatography in |
| | | | | 20 mg scale. |

### Example 2 Solubility Experiments

Solubility testing was performed with the following test items and conditions:
Test items: axitinib (99.99% purity by HPLC);
   axitinib-N-mPEG-succinoyloxymethyl prodrug (93.6% purity by HPLC)
Test media: phosphate buffer saline pH 7.4
Incubation condition: 24 hour at 22 °C with continuous shaking
Test concentration: 1 mg/mL
Data analysis: Solubility of test item was determined by HPLC analysis with calibration curve

The HPLC conditions were as follows:

| HPLC | PCR-HPLC-003 (Shimadzu) |
|---|---|
| Column Specification | Kinetex, C18, 100 × 4.6 mm, 2.6 μM |
| | particle size, 100Å |
| Mobile Phase A | 0.05% Acetic Acid in Ultrapure Water |
| | Type-I |
| Mobile Phase B | Acetonitrile (100%) |
| Flow Rate | 1.0 mL/min |
| Run Time (Minutes) | 18 |
| Column oven Temperature (°C) | 40 |
| Calibration Range (μg/mL) | 0.78-200 |
| Axitinib | |
| Calibration Range (μg/mL) N- | 0.78-100 |
| mPEG-succinoyloxymethyl | |
| Retention time of Analyte | ~7.2 |
| (Minutes) Axitinib | |
| Retention time of Analyte | ~ 8.0 |
| (Minutes) N-mPEG- | |
| succinoyloxymethyl | |
| Lamda Max | 225 nm |
| Autosampler Temperature (°C) | 4 |
| Injection Volume (AL) Axitinib | 10 |
| Injection Volume (AL) N- | 5 |
| mPEG-succinoyloxymethyl | |

The results are as follows:

| Test Item | Solubility (μg/mL) |
|---|---|
| Axitinib | <0.78 |
| N-mPEG | 99.37 |
| succinoyloxymethyl | |
| Prodrug | |

The literature reported solubility of axitinib is about 0.2 mcg/mL. The results demonstrate that the solubility of the prodrugs was enhanced by ~500× for N-mPEG Oxymethyl Prodrug.

### Example 3 (Prophetic)

Conversion of axitinib-N-mPEG-succinoyloxymethyl prodrug to axitinib is set forth below.

### Methods

Each prodrug, at one concentration (1 μM in the final incubation), is incubated with hrCESs (a combination of hrCES-1 and hrCES-2, 0.1 mg protein/mL per hrCES) in phosphate buffer (100 mM, pH 7.4) containing MgCl2 (5 mM). The incubation mixture is equilibrated in a shaking water bath at 37 °C for 5 minutes. The reaction is initiated by the addition of the prodrug, followed by incubation at 37 °C. Aliquots of the incubation solutions are sampled at 0, 15, 30, 60, and 120 minutes. The reaction is terminated by the addition of 50% ice-cold acetonitrile (ACN)/0.1% formic acid containing an internal standard (IS, 0.2 μM metoprolol or 0.2 μM tolbutamide for the positive or negative ionization mode in mass spectrometry, respectively).

After the removal of protein by centrifugation at 1,640 g (3,000 rpm) for 10 minutes at 4 °C, the supernatants are transferred to an HPLC autosampler plate and stored at −20 °C until analysis. The remaining prodrug (expressed as the peak area ratio of prodrug to IS) and the formation of the acid product per prodrug (the final hydrolysis product of each prodrug) are determined by LC-MS/MS (Appendix 1). CES activities of the hrCESs used in this study are verified in parallel by determining the time-dependent formation (0, 3, 5, and 10 minutes) of PNP based on absorbance at 410 nm using a non-specific esterase probe substrate, PNPB, at 1 mM. The experimental conditions for CES reaction phenotyping and sample analysis are summarized below.

Conditions for CES Reaction Using hrCESs and Sample Analysis

| | |
|---|---|
| Incubation | TA: 1 μM; |
| Composition | hrCESs: hrCES-1 + hrCES-2, 0.1 mg protein/mL per |
| | hrCES; |
| | Phosphate buffer: 100 mM, pH 7.4; |
| | MgCl2: 5 mM |
| Equilibration | 37 °C for 5 min |
| Incubation | 37 °C for 0, 15, 30, 60, and 120 min (n = 1) |
| Time | |
| Reaction | 50% ACN/0.1% formic acid containing IS (1:4, |
| Termination | sample:crash solvent, v/v) |
| Sample | Centrifugation at 1,640 g (3,000 rpm) for 10 minutes |
| Treatment | at 4 °C |
| Sample | LC-MS/MS |
| Analysis | |

### Data Analysis

The percent remaining of the prodrug is calculated using the following equation:

% Remaining of prodrug=100×At/A0

Where, At is the peak area ratio (prodrug to IS) at time t and A0 is the peak area ratio (prodrug to IS) at time zero.

The elimination rate constant of prodrug is estimated from first-order reaction kinetics: Ct=C0·e−kt

Where, C0 and Ct are the concentrations of the prodrug (expressed as the peak area ratios of prodrug to IS) at time zero and incubation time t (min) and k is the elimination rate constant (min-1).

The elimination half-life of the prodrug (if applicable) is calculated using the following equation before the plot starts to plateau:

t½=0.693/k

Where, t½ is the half-life (min) and k is the elimination rate constant (min-1).

The in vitro intrinsic clearance of the prodrug (if applicable) is calculated using the following equation:

CLint=k/P

Where, CLint is the in vitro intrinsic clearance, k is the elimination rate constant (min-1), and P is the enzyme concentration in the incubation medium (mg protein/mL).

All intrinsic clearance parameters are estimated using GraphPad® Prism (GraphPad Software, San Diego, CA, USA) and Microsoft Office Excel (Microsoft Corporation, Redmond, WA, USA).

### Example 4 (Prophetic)

Test articles are prepared in suspension in a mixture of DMSO (5%) and 0.5%-CMC-Na (95%, v/v), at a concentration of 3 mg/ml axitinib-molar-equivalence for an inventive prodrug. Male ICR mice (64, body weight ranging 18-22 g) are grouped randomly in 4 groups (16 animals per group). Test articles are administered to animals through oral-gavage, at a dose of molar-equivalent to 30 mg/kg of axitinib, after the animals were fasted for 12 h. Blood samples are collected from orbit to heparinized EP tube at time points of 0.25, 0.5, 1, 2, 4, 6, and 8 h following the administration of the dosing solution. Blood samples are centrifuged at 5,000 rpm and 4 °C for 10 min, and plasma samples are collected and kept at −80 °C. Sample analysis: Plasma sample (10 μL) is mixed well with acetonitrile (110 μL). The sample is then centrifuged at 12,000 rpm and 4 °C. The supernatant is analyzed with an LC-MS/MS instrument, and the target analytes are axitinib and its corresponding prodrug molecules.

### Example 5 (Prophetic)

Male ICR mice (body weight: 18 to 22 g) are divided into 6 groups randomly with 6 animals per group, with blood sample collection from 6 animals at each time point for a total of 6 time points. Dosing solution of a test article is prepared by dissolving or suspending a compound in a solvent system. For all the compounds, the concentration of dosing solution is 3 mg/mL of axitinib molar equivalent, and the dose is 30 mg/kg of axitinib molar equivalent. Animals are fasted for 12 h, and then given the test article in dosing media at a dosing volume calculated according to the above information. After dosing, blood samples are collected at the pre-set time points of 0.5, 1, 2, 4, 6, and 8 h. Blood samples are centrifuged at 5,000 rpm and 4 °C for 10 min, and plasma samples are collected and kept at =80 °C. Sample analysis: Plasma sample (20 μL) is mixed well with acetonitrile (220 μL). The sample is then centrifuged at 12,000 rpm and 4 °C. The supernatant is analyzed with an LC-MS/MS instrument, and the target analytes.

### Example 6

### axitinib-N-mPEG₂-oxymethyl prodrug

The compound of Example 6 was prepared according to the scheme below:

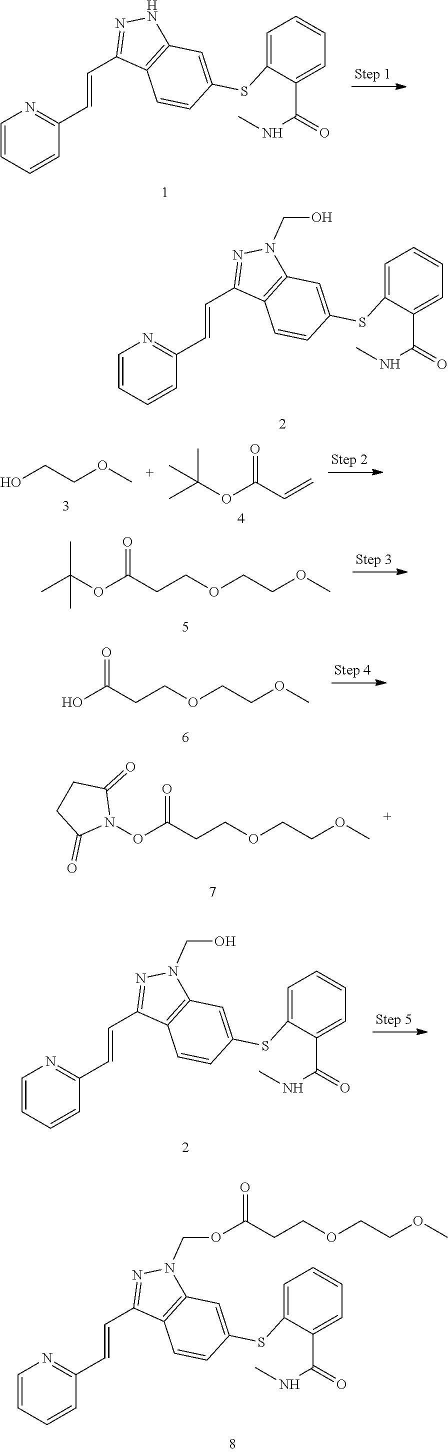

The sample number, batch size, conditions, yield and discussion are set forth below for each step of the process:

### Step 1

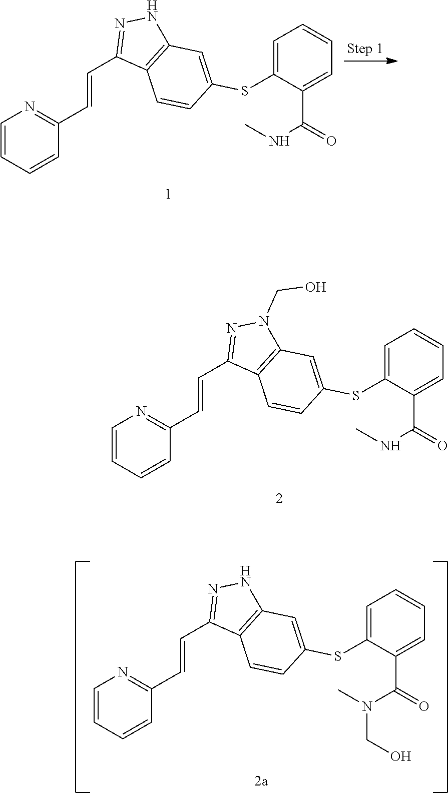

| | | | | |
|---|---|---|---|---|
| | Batch | | | |
| S. No | size | Conditions | Yield | Comments |

Step-1

| | | | | |
|---|---|---|---|---|
| 1 | 0.5 g | 37% formaldehyde | 0.43 g | Formation of compound |
| | (1) | (3.0 eq.), TEA (3.0 | (80%) | 2 was confirmed by |
| | | eq.), DMF, 0 °C, 3 h. | | ¹H-NMR. |
| 2 | 1.0 g | 37% formaldehyde | 0.9 g | 1H NMR indicates the |
| | | (3.0 eq.), TEA (3.0 | (80%) | compound 2 along |
| | | eq.), DMF, 0 °C, 3 h. | | with traces of |
| | | | | Regiosiomer (2a). |
| 3 | 5 g + | 37% formaldehyde | 20 g | 1H NMR indicates the |
| | 10 g + | (3.0 eq.), TEA (3.0 | (90%) | compound 2 along |
| | 5 g | eq.), DMF, 0 °C, 3 h. | | with traces of |
| | | | | regiosiomer (2a). |

### Steps 2-5

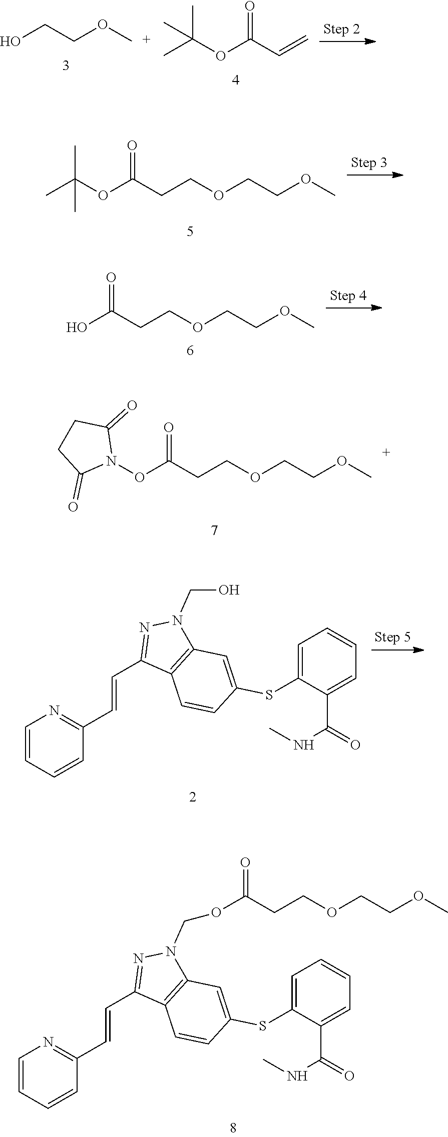

### Step 2

| | | | | |
|---|---|---|---|---|
| | Batch | | | |
| S. No | size | Conditions | Yield | Comment |

Step-2

| | | | | |
|---|---|---|---|---|
| 1 | 2.0 g | t-Butyl acrylate(3.0 | 2.6 g | Formation of compound |
| | (3) | eq.), Triton-B (40% | (48%) | 5 was confirmed by |
| | | in methanol) (0.4 | | ¹H-NMR. |
| 2 | 30 g | eq.), 0 °C-RT, 3 h. | 45 g | Isolated compound 5 by |
| | (3) | | (55%) | silica gel column |
| | | | | chromatography in 47.6 g |
| | | | | scale. |

### Step 3

| | | | | |
|---|---|---|---|---|
| | Batch | | | |
| S. No | size | Conditions | Yield | Comments |

Step-3

| | | | | |
|---|---|---|---|---|
| 1 | 2.5 g | Triflouroacetic acid | 1.63 g | Formation of compound |
| | (5) | (5 V), DCM (5 V), | (90%) | 6 was confirmed by |
| | | 0 °C-RT, 2 h. | | ¹H-NMR. |
| 2 | 45 g | Triflouroacetic acid | 30 g | Isolated compound 6 by |
| | (5) | (5 V), DCM (5 V), | (91%) | silica gel column |
| | | 0 °C-RT, 2 h. | | chromatography in 1.63 g |
| | | | | scale. |
| | | | | Isolated compound 6 by |
| | | | | silica gel column |
| | | | | chromatography in 30 g |
| | | | | scale. |

### Step 4

| | | | | |
|---|---|---|---|---|
| | Batch | | | |
| S. No | size | Conditions | Yield | Comments |

Step-4

| | | | | |
|---|---|---|---|---|
| 1 | 0.5 g | N-Hydroxysuccinimide | 0.150 g | Formation of compound |
| | (6) | (1.1 eq.), | | 7 was confirmed by |
| | | DIC (1.0 eq.), DCM, | | ¹H-NMR. |
| | | 0 °C-RT, 16 h | | Crude 500 mg |
| 2 | 3 g | N-Hydroxysuccinimide | 1.8 g | purification done |
| | (6) | (1.1 eq.), | (43.4%) | 150 mg isolated. |
| | | DIC (1.0 eq.), DCM, | | 1.8 g confirmed by |
| | | 0 °C-RT, 16 h | | ¹H-NMR. |
| 3 | 5 g + | N-Hydroxysuccinimide | 14.4 g | 14.4 g Conformed by |
| | 15 g | (1.1 eq.), | (43.4%) | ¹H-NMR. |
| | (6) | DIC (1.0 eq.), DCM, | | |
| | | 0 °C-RT, 16 h | | |

### Step 5

| | | | | |
|---|---|---|---|---|
| | Batch | | | |
| S. No | size | Conditions | Yield | Comments |

Step-5

| | | | | |
|---|---|---|---|---|
| 1 | 0.9 g + | 7 (1.2 eq), TEA (2.0 | 10.5 g | 10.5 g isolated after |
| | 4.5 g + | eq), DMF | | purification. |
| | 15 g | 0 °C-RT, 16 h. | | confirmed by 1H NMR & |
| | | | | HPLC: 98.8%. |
| | | | | After micronization 3 g |
| | | | | isolated |
| | | | | Confirmed by 1H NMR, |
| | | | | HPLC: 98.3%. |

The micronization of the axitinib-N-m (PEG)₂-oxymethyl prodrug obtained in Example 6 in a dry dispersion was D90=12.4 μm; D50=3.6 μm and D10=0.6 μm and dispersed in water was D90=12.2 μm; D50=5.2 μm and D10=1.1 μm.

### Example 7

Axitinib prodrugs of the present invention were tested for solubility in phosphate buffer saline (PBS, pH-7.40). The results are set forth in the table below:

| S. No. | Test Item Name | Solubility (μg/ml) |
|---|---|---|
| 1 | Axitinib Prodrug | Not Soluble |
| 2 | Axitinib-N-Succinoyloxymethyl Prodrug | 217.44 |
| 3 | Axitinib-N-mPEG Oxymethyl Prodrug | 99.37 |
| 4 | Axitinib-N-m(PEG)1-oxymethyl prodrug | Not Soluble |
| 5 | Axitinib-N-m(PEG)2-oxymethyl prodrug | 1.10 |
| 6 | Axitinib-N-m(PEO)3-oxymethyl prodrug | 6.16 |

### Example 8

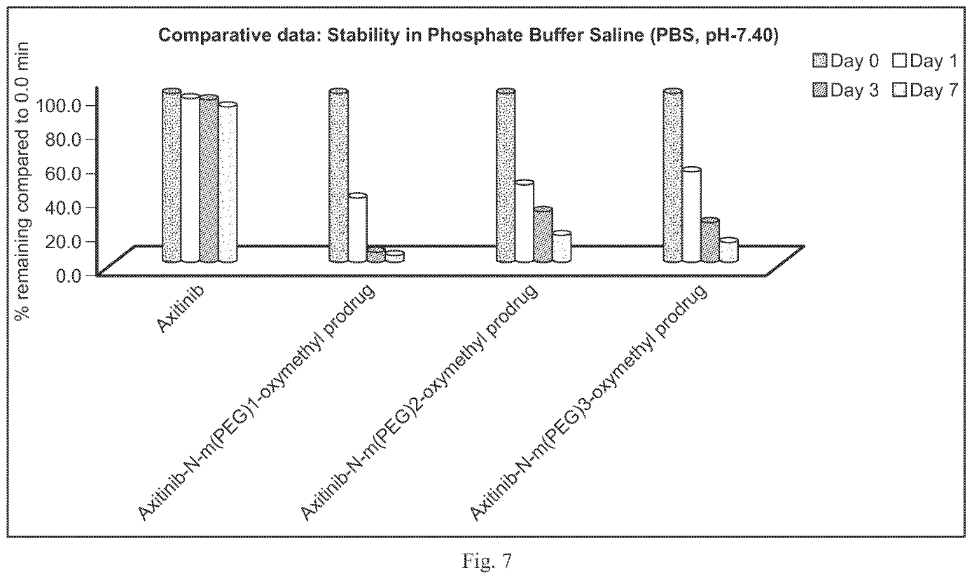

Axitinib prodrugs of the present invention were tested for stability in phosphate buffer saline (PBS, pH-7.40). The results are set forth in the table below and FIG. 7:

% remaining compared to Day 0

| Test/Control item | Day 0 | Day 1 | Day 3 | Day 7 |
|---|---|---|---|---|
| Axitinib | 100.00 | 96.72 | 96.25 | 91.78 |
| Axitinib-N-m(PEG)1-oxymethyl prodrug | 100.00 | 38.78 | 6.70 | 5.00 |
| Axitinib-N-m(PEG)2-oxymethyl prodrug | 100.00 | 45.85 | 30.48 | 16.86 |
| Axitinib-N-m(PEG)3-oxymethyl prodrug | 100.00 | 53.90 | 24.04 | 12.70 |

### Example 9

The purity by HPLC for axitinib prodrugs of the present invention is set forth below:

| | | Purity by | Purity by |
|---|---|---|---|
| | | HPLC | HPLC |
| SI.No | Name & Target structure | (Run 1) | (Run 2) |
| 1 | 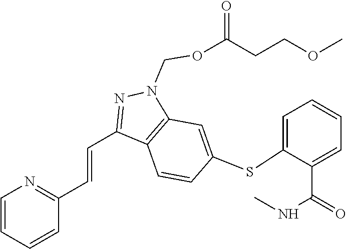 [SMILES: CNC(=O)c1ccccc1Sc1ccc2c(/C=C/c3ccccn3)nn(COC(=O)CCOC)c2c1] | 99.34% | 98.80% |
| | Axitinib-N-m(PEG)1-oxymethyl prodrug | | |
| 2 | 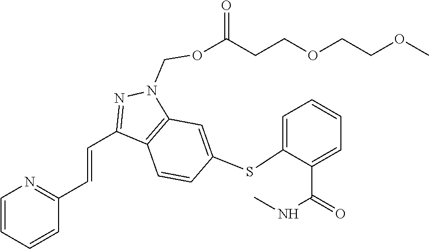 [SMILES: CNC(=O)c1ccccc1Sc1ccc2c(/C=C/c3ccccn3)nn(COC(=O)CCOCCOC)c2c1] | 99.14% | 99.14% |
| | Axitinib-N-m(PEG)2-oxymethyl prodrug | | |
| 3 | 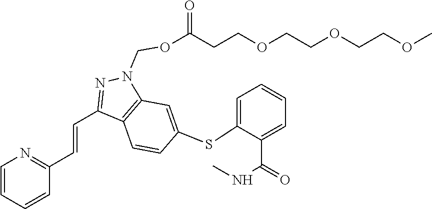 [SMILES: CNC(=O)c1ccccc1Sc1ccc2c(/C=C/c3ccccn3)nn(COC(=O)CCOCCOCCOC)c2c1] | 94.90% | 94.44% |
| | Axitinib-N-m(PEG)3-oxymethyl prodrug | | |

1. A compound of formula I: 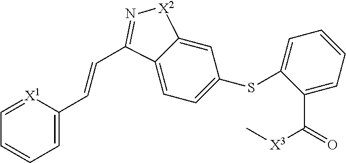
wherein:
X¹ is selected from N or N⁺Y¹;
X² is selected from NH or NY²;
X³ is selected from NH or NY³;
Y¹ is selected from —CH₂OCO(OCH₂CH₂)n¹OM¹; or —CH₂OCO(CH₂CH₂O)n^{1a}Z¹; or —CH₂OCO(CH₂)n^{1b}COOH;
Y² is selected from —CH₂OCO(OCH₂CH₂)n²OM²; or —CH₂OCO(CH₂CH₂O)n^{2a}Z²; or —CH₂OCO(CH₂)n^{2b}COOH;
Y³ is selected from —CH₂OCO(OCH₂CH₂)n³OM³; or —CH₂OCO(CH₂CH₂O)n^{3a}Z³; or —CH₂OCO(CH₂)n^{3b}COOH;
n¹, n^{1a}, n^{1b}, n² n^{2a}, n^{2b}, n³, n^{3a} and n^{3b} are independently 0 or an integer from 1 to 8;
M¹, M², M³, Z¹, Z² and Z³ are independently selected from H, optionally substituted C₁₋₆alkyl and optionally substituted aryl;
wherein at least one of X¹, X² and X³ is not N or NH;
wherein at least one of Y¹, Y² or Y³ are the respective —CH₂OCO(CH₂CH₂O)nZ;
and pharmaceutically acceptable salts thereof.

2. The compound of claim 1, wherein:
X¹ is N⁺Y¹;
X² is NH; and
X³ NH; and
Y¹ is —CH₂OCO(CH₂CH₂O)n¹Z¹.

3. The compound of claim 1, wherein:
X¹ is N;
X² is NY²;
X³ is NH; and
Y² is —CH₂OCO(CH₂CH₂O)n²Z².

4. The compound of claim 1, wherein:
X¹ is N;
X² is NH;
X³ is NY³; and
Y³ is —CH₂OCO(CH₂CH₂O)n³Z³.

5. The compound of claim 1 wherein n¹, n² or n³ is 2.

6. The compound of claim 1 wherein n^{1a}, n^{2b} or n^{3a} is 2.

7. The compound of claim 1 wherein n^{1b}, n^{2b} or n^{3b} is 2.

8. A pharmaceutical composition of claim 1 comprising a compound of formula I and a pharmaceutically acceptable excipient.

9. The pharmaceutical composition of claim 8, in the form of an oral solid dosage form.

10. The pharmaceutical composition of claim 9, in the form of a tablet.

11. The pharmaceutical composition of claim 8, in the form of an ocular formulation.

12. The pharmaceutical composition of claim 11, in the form of an implant, an injection, a solution, a suspension or an ointment.

13. A method of treating a disease or condition comprising administering a compound of claim 1.

14. The method of claim 13, wherein the disease or condition is advanced renal cell carcinoma.

15. The method of claim 13, wherein the disease or condition state is an ocular disease or condition.

16. The method of claim 15, wherein the ocular disease or condition is AMD, DME, or RVO.

17. The method of claim 1, wherein the compound of formula I is converted in-vivo to a compound of formula II

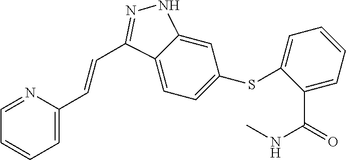 [SMILES: CNC(=O)c1ccccc1Sc1ccc2c(/C=C/c3ccccn3)n[nH]c2c1]

18. A method of treating a disease or condition by axitinib therapy comprising administering a compound of claim 1.

19. A hydrogel comprising a compound claim 1.

20. A xerogel comprising a compound of claim 1.

21-36. (canceled)
